(19) United States
(12) Patent Application Publication
(10) Pub. No.: US 2022/0190257 A1
(43) Pub. Date: Jun. 16, 2022

(54) FUNCTIONAL FILM, METHOD FOR FORMING SAME, AND ORGANIC ELECTROLUMINESCENT ELEMENT

(71) Applicant: Konica Minolta, Inc., Tokyo (JP)

(72) Inventors: Mayuka KABASAWA, Hamura-shi, Tokyo (JP); Yuko IKEDA, Toyohashi-shi, Aichi (JP); Miyuki OKANIWA, Sagamihara-shi, Kanagawa (JP); Hiroshi KITA, Hachioji-shi, Tokyo (JP)

(21) Appl. No.: 17/440,353

(22) PCT Filed: Mar. 6, 2020
(86) PCT No.: PCT/JP2020/009624

§ 371 Date: Sep. 17, 2021

(30) Foreign Application Priority Data
Mar. 19, 2019 (JP) 2019-050713

(51) Int. Cl. H01L 51/00; C07D 209/86; C09K 11/06; C07D 471/04

(52) U.S. Cl. CPC H01L 51/0072; C07D 209/86; H01L 51/5016; C07D 471/04; C09K 11/06

(57) ABSTRACT

A functional film includes an aromatic compound which has a condensed or noncondensed 6-membered aromatic hydrocarbon ring or aromatic heterocyclic ring having four or more condensed aromatic ring groups containing not less than 14Π electrons, wherein three or more of the condensed aromatic ring groups containing not less than 14Π electrons are adjacent as substituents. For the aromatic compound, a film density value calculated by molecular dynamics calculation of NPT ensemble at 300 K is defined as an initial film density of the functional film comprising only the aromatic compound. For the aromatic compound, when a film density value calculated by molecular dynamics calculation at 370 K is defined as a film density value after storage of the functional film at the temperature, the difference between the initial film density and the film density value after storage is 1% or less with respect to the initial film density.

### TECHNICAL FIELD

The present invention relates to a functional film, a method for forming the same, and an organic electroluminescence element. More specifically, the present invention relates to a functional film, etc., having excellent low voltage driveability, high luminous efficiency, long service life, resistance to drive voltage fluctuation, and vapor deposition reproducibility, without burning of a vapor deposition boat.

### BACKGROUND ART

In order to form a functional film constituting, an organic electroluminescence element (hereinafter, also referred to as “organic EL element”) by a vapor deposition method, the compound contained in the functional film is required for a high glass transition temperature (Tg) thereof for improving thermal stability and inhibiting changes in film quality and crystallization upon drive. However, the high Tg necessitates higher molecular weights and more π-conjugated systems. As π-conjugated system increases, the π-π interaction also increases, which will raise the sublimation temperature and decompose the material.

In order to inhibit the π-π interaction, compounds that introduce steric hindrance groups or have multiple conformations have been proposed, but their effects are insufficient or the sublimation temperature is increased due to the high molecular, which has not yet been resolved.

Further, the inhibition of the π-π interaction also inhibits the intermolecular interaction of the highest occupied molecular orbital (HOMO) and the lowest unoccupied molecular orbital (LUMO), which causes a problem of lowering carrier transportability.

As described above, element fabrication by using the vapor deposition method has the trade-off relationship between the improvement of stability due to the increase in Tg (higher molecular weight) and the vapor depositionability.

For example, in the technique disclosed in Patent Literature 1, an attempt has been made to inhibit intermolecular interactions by using a compound containing a large number of conformations being capable of having various three-dimensional structures, but it is not a suitable solution for vapor deposition because there are problems of the vapor deposition temperature rise due to high molecular weights and reduction of transportability⁻ due to the decrease in the volume fraction of the charge transport sites.

The technique disclosed in Patent Literature 2 has been known as a technique that enables to minimize deterioration upon drive or at elevated temperature storage by defining the film density of the organic layer of the organic EL element. Further, Patent Literature 3 discloses a technique in which a film density close to that by vapor deposition can be obtained by heating while applying tension upon drying in element fabrication with a wet process.

However, there is no description of the problem that the film density changes with an elapsed time and the film quality changes upon drive of storage, etc. Since the element performance deteriorates due to such a change in the film condition with an elapsed time, it is required for further improvement.

On the other hand, Patent Literature 4 discloses a technique that increases an entropy by possessing chirality in the molecule and improves stability by inhibition of film quality fluctuation/crystallization. However, when the compound possessing chirality and being capable of forming a plurality of atropisomers is vapor-deposited, there occurs a problem of isomerization due to heat and the reduction of reproducibility, which, however, is not described in the above literature.

Moreover, Patent Literature 5 describes a compound in which an aromatic heterocyclic ring having 6 π electrons or 10π electrons is adjacently bonded to a benzene ring. However, these compounds have low steric hindrance, and changes such as upon film formation and drive/storage with an elapsed time are insufficiently inhibited, which has been required for further improvement.

### CITATION LIST

### Patent Literature

### [Patent Literature 1]

WO2018/123783

### [Patent Literature 2]

WO2007/020718

### [Patent Literature 3]

WO2011/114870

### [Patent Literature 4]

JP 2014-229721A

### [Patent Literature 5]

JP 2006-00394A

### SUMMARY OF INVENTION

### Technical Problem

The present invention has been made in view of the aforementioned problems and situations, and the solution to these problems is to provide a functional film having low voltage driveability, high luminous efficiency, long service life, excellent resistance to drive voltage fluctuation, and excellent vapor deposition reproducibility, without burning of a vapor deposition boat, a method for forming the same, and an organic electroluminescence element.

### Solution to Problem

The present inventors have investigated the causes and the like of the aforementioned problems in order to achieve the above object, and have found that a functional film comprising an aromatic compound having a specific structure (hereinafter may be referred to as “aromatic compound having polysubstituted structures”), which is a functional film having excellent low voltage driveability, high luminous efficiency, long service life, resistance to drive voltage fluctuation, and vapor deposition reproducibility, without burning of a vapor deposition boat, owing to the functional film in which a difference between an initial film density value defined as a film density calculated by molecular dynamics calculation of NPT ensemble and a film density value after the storage, is in the specific range with respect to the initial film density.

Namely, the aforementioned problems can be solved by the following means.

1. A functional film comprising an aromatic compound, wherein the aromatic compound has a condensed or noncondensed 6-membered aromatic hydrocarbon ring or aromatic heterocyclic ring having four or more condensed aromatic ring groups containing not less than 14 π electrons, wherein three or more of the condensed aromatic ring groups containing not less than 14 π electrons are adjacent to one another as substituents, and Wherein

for the aromatic compound, when a film density value calculated by molecular dynamics calculation of NPT ensemble at 300 K is defined as an initial film density of the functional film comprising only the aromatic compound, and

for the aromatic compound, when a film density value calculated by molecular dynamics calculation at 370 K is defined as a film density value after storage of the functional film at the temperature, the difference between the initial film density and the film density value after storage is 1% or less with respect to the initial film density.

2. The functional film according to item 1, wherein the initial film density value is in the range of 1.00 to 1.20 g/cm³.

3. The functional film according to item 1 or 2, wherein the aromatic compound has a chirality generation site.

4. The functional film according to any one of items 1 to 3, wherein the aromatic compound has a structure represented by the following general formula (1):

[Chemical Formula 1]

Ar [symbol] HetAr)ₙ General formula (1)

wherein Ar represents substituted or unsubstituted heteroaryl, or substituted or unsubstituted aryl; HetAr represents a substituted or unsubstituted condensed aromatic heterocyclic group containing not less than 14 π electrons; and n represents an integer of 4 or more.

5. The functional film according to any one of items 1 to 4, wherein the aromatic compound has a structure represented by the general formula (2):

[Chemical Formula 2]

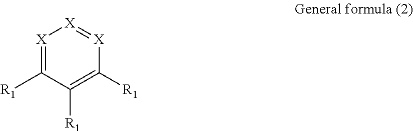

wherein X represents N or CR₂ and at least one X represents CR₂; R₁ represents a condensed aromatic heterocyclic group containing not less than 14π electrons; and R₂ represents a hydrogen atom or R₁, or represents any group selected from the group consisting of an alkyl group, a cycloalkyl group, an alkenyl group, an aromatic hydrocarbon group, an aromatic heterocyclic group, a heterocyclic group, an alkoxy group, a cycloalkoxy group, an aryloxy group, an alkylthio group, a cycloalkylthio group, an arylthio group, an aryloxycarbonyl group, a sulfamoyl group, an alkoxycarbonyl group, an acyl group, an acyloxy group, an amide group, a carbamoyl group, a ureido group, a sulfinyl group, an alkylsulfonyl group, an arylsulfonyl group or a heteroarylsulfonyl group, an amino group, a halogen atom, a fluorinated hydrocarbon group, a cyano group, a nitro group, a hydroxy group, a mercapto group, a silyl group, and a phosphono group, provided that at least one R₂ represents R₁.

6. The functional film according to any one of items 1 to 5, wherein the condensed aromatic heterocyclic group containing not less than 14π electrons has a nitrogen (N) atom.

7. The functional film according to any one of items 1 to 6, wherein the condensed aromatic heterocyclic group containing not less than 14π electrons has at least two nitrogen (N) atoms.

8. The functional film according to any one of items 1 to 7, wherein the aromatic compound has five or less condensed aromatic ring groups containing not less than 14π electrons.

9. The functional film according to item 5, wherein in the compound having the structure represented by the general for (2), R₂ represents R₁, or represents any group selected from the group consisting of a cycloalkyl group, an aromatic hydrocarbon group, an aromatic heterocyclic group, an amino group, a fluorinated hydrocarbon group, or a nitro group, a silyl group and a phosphono group.

10. The functional film according to any one of items 1 to 9, wherein the aromatic compound has a molecular weight in the range of 1,000 to 2,000.

11. The functional film according to any one of items 1 to 10, comprising the aromatic compound in an amount of 50% by weight or more.

12. The functional film according to any one of items 1 to 11, being a charge transport film.

13. A method for forming the functional film according to any one of items 1 to 12 by a vacuum vapor deposition method.

14. An organic electroluminescence element comprising at least a pair of electrodes and one or a plurality of layers, wherein at least one layer of the plurality of layers has the functional film according to any one of items 1 to 12.

15. The organic electroluminescence element according to item 14, wherein at least two adjacent layers among the plurality of layers are each the functional film according to any one of items 1 to 12.

### Advantageous Effects of Invention

The aforementioned means of the present invention provides a functional film having excellent low voltage drive, high luminous efficiency, long service life, resistance to drive voltage fluctuation, and vapor deposition reproducibility, without burning of a vapor deposition boat, a method for forming the same, and an organic electroluminescence element.

The mechanism of exhibition or mechanism of action of the effect of the present invention has not been clarified, but it is inferred as follows.

In the present invention, by using the aromatic compound having a specific polysubstituted structure, a functional film in which the movement of each aromatic group is inhibited and the change in film density upon drive or storage is small, can be provided. In other words, an aromatic compound having high stability and being suitable for vapor deposition can be provided because the compound can inhibit aggregation, etc., due to its little π-π interaction and therefore can sublime without decomposition even if the molecular weight is increased, minimizing changes in the molecular condition in a state of film formation.

Furthermore, in the present invention, since the aromatic compound is a mixture of atropisomers, due to its entropy-increasing effect, a stable amorphous film can be formed even upon continued energization and under elevated temperature storage by inhibiting the molecular fluctuation caused by further film quality fluctuation/crystallization inhibition, and improvement on the luminous efficiency and the service life of the light emitting element can be contemplated. In the case of using the mixture of atropisomers, there is a problem of isomerization due to heating upon vapor deposition and causing change in the mixing ratio of the isomers, lowering the vapor deposition reproducibility. However, in the present invention, an element that hardly undergoes isomerization and is favorable in reproducibility can be provided due to the larger steric hindrance by the polysubstituted structure and inhibition of the increase in the vapor deposition temperature.

### BRIEF DESCRIPTION OF DRAWINGS

FIG. 1 is a schematic diagram of a lighting device.

FIG. 2 is a schematic view of a lighting device.

### DESCRIPTION OF EMBODIMENTS

The functional film of the present invention is a functional film comprising an aromatic compound, wherein the aromatic compound has a condensed or noncondensed 6-membered aromatic hydrocarbon ring or aromatic heterocyclic ring having four or more condensed aromatic ring groups containing not less than 14π electrons, wherein three or more of the condensed aromatic ring groups containing not less than 14π electrons are adjacent to one another as substituents, and wherein, for the aromatic compound, when a film density value calculated by molecular dynamics calculation of NPT ensemble at 300 K is defined as an initial film density of the functional film comprising only the aromatic compound, and for the aromatic compound, when a film density value calculated by molecular dynamics calculation at 370 K is defined as a film density value after storage of the functional film at the temperature, the difference between the initial film density and the film density value after storage is 1% or less with respect to the initial film density. This feature is a technical feature common to or corresponding to the following embodiments.

In an embodiment of the present invention, from the viewpoint of exhibiting the effect of the present invention, the initial film density value is preferably in the range of 1.00 to 1.20 g/cm³ in terms of charge transportability and inhibition of the change in film quality with an elapsed time.

Further, the aromatic compound having a chirality generation site is preferred as an entropy-increasing effect by increasing the number of isomers in terms of enhancing the stability of the charge transfer/light emitting thin film.

The aromatic compound having the structure represented by the general formula (1) is preferred from the viewpoint of charge transportability.

Further, the aromatic compound having the structure represented by the general formula (2) is preferred from the viewpoint of improving stability with an elapse time.

The condensed aromatic heterocyclic group containing not less than 14π electrons preferably has at least a nitrogen (N) atom from the viewpoint of improving charge transportability.

Further, the condensed aromatic heterocyclic group containing not less than 14π electrons preferably has at least two nitrogen (N) atoms from the viewpoint of improving charge transportability.

Further, the aromatic compound preferably has five or less condensed aromatic ring groups containing not less than 14π electrons from the viewpoint of improving charge transportability.

In the compound having the structure represented by the general formula (2), R₂ preferably represents R₁, or any group selected from the group consisting of a cycloalkyl group, an aromatic hydrocarbon group, an aromatic heterocyclic group, an amino group, a fluorinated hydrocarbon group, a nitro group, a silyl group and a phosphono group, from the viewpoint of stability with an elapsed time.

A molecular weight of the aforementioned compound is preferably in the range of 1,000 to 2,000 from the viewpoint of improving the stability of the compound. Being in the range of 1,000 to 1,500 is more preferred from the viewpoint of the compound stability and the inhibition of occurrence of burning after vapor deposition.

The functional film of the present invention preferably comprises the aforementioned compound in an amount of 50% by weight or more from the viewpoint of improving stability with an elapsed time.

The functional film of the present invention is preferably a charge transport film from the viewpoint of improving charge transportability.

The method for forming the functional film of the present invention is characterized in that it is formed by a vacuum vapor deposition method.

The organic electroluminescence element of the present invention is an organic electroluminescence element comprising at least a pair of electrodes and one or a plurality of layers, wherein at least one layer of the plurality of layers has the functional film of the present invention.

Among the plurality of layers, at least two adjacent layers are preferably the functional films of the present invention from the viewpoint of improving charge transportability.

Hereinafter, the present invention, the constituent elements thereof, and embodiments and aspects for carrying out the present invention will be described in detail. As used herein, the numerical values described before and after “to” are included as the lower limits and the upper limits, respectively.

### Summary of Functional Film of Present Invention

The functional film of the present invention is a functional film containing an aromatic compound, wherein the aromatic compound has a condensed or noncondensed 6-membered aromatic hydrocarbon ring or aromatic heterocyclic ring having four or more condensed aromatic ring groups containing not less than 14π electrons, wherein three or more of the condensed aromatic ring groups containing not less than 14π electrons are adjacent to one another as substituents, and wherein, for the aromatic compound, when a film density value calculated by molecular dynamics calculation of NPT ensemble at 300 K is defined as an initial film density of the functional film comprising only the aromatic compound, and for the aromatic compound, when a film density value calculated by molecular dynamics calculation at 370 K is defined as a film density value after storage of the functional film at the temperature, the difference between the initial film density and the film density value after storage is 1% or less with respect to the initial film density.

In the present invention, “molecular dynamos calculation of NPT ensemble” is as follows.

“Molecular dynamics calculation of NPT ensemble” is also called “molecular dynamics simulation”, which is a method of virtually arranging atoms and molecules on a computer and investigating their motion. in actual molecular dynamics simulations, the temperature and pressure are often maintained constant for comparison with experiments. For that purpose, the motion equation of Newton is rewritten and the simulation is carried out with canonical ensemble (NVT ensemble) under a constant temperature, or constant temperature and constant pressure ensemble (NPT ensemble) under a constant temperature and pressure, by controlling the kinetic energy and volume.

In the present invention, “molecular dynamics calculation of NPT ensemble” is adopted, and the following initial film density and the ratio of change in film density after storage are calculated.

(1) Initial Film Density

A density value calculated by the molecular dynamics calculation of NPT ensemble at 300K is used as the initial film density of the functional film comprising only the compound.

In the present invention, the initial film density value is preferably in the range of 1.00 to 1.20 g/cm³. Furthermore, from the viewpoint of maintaining charge transportability, it is preferably 1.05 g/cm³ or more. Moreover, it is preferably 1.15 g/cm³ or less and more preferably 1.10 g/cm³ or less, in order to inhibit aggregation and improve stability.

(2) Ratio of Change in Film Density After Storage

When the film density value of the functional film calculated by carrying out the molecular dynamics calculation under the condition of 370 K is used as the film density value after storage of the functional film having stored under the temperature, the difference between the initial film density and the film density after storage is calculated. From that value, as shown below, the percentage with respect to the initial film density is obtained and used as a measure of the ratio of change in film density.

Ratio of change in film density (%){|initial film density−film density after storage|initial film density}×100

### <Measurement Method of Film Density >

The film density specified in the present invention is calculated and obtained by the following method.

### <Calculation Software>

Materials Science Suite (manufactured by Schrodinger K.K.)

### <Calculation Procedure>

(1) A molecular structure is created and the structure is optimized.
(2) By using the structure optimized in (1), an amorphous structure having an initial film density of 0.5 g/cm³ is produced.
(3) By using the following molecular dynamics (MD) calculation conditions, the amorphous structure created in (2) is equilibrated.
(4) The film density (g/cm³) in the obtained cell is determined so as to match the following conditions. The term “cell” as referred to in the present invention refers to a unit comprising a specified number of molecules.

<MD Calculation Conditions>

The calculation time was 10 nanoseconds, the ensemble method was NPT, the pressure was 1 atm, and the number of molecules was 300 molecules. The temperature was 300 K or 370 K.

<Calculation Conditions for Film Density>

For the film density specified in the present invention, when the calculation for 10 nanoseconds was completed, the average value of the fiscal 20% of the trajectory data was taken as the film density. At this time, the standard deviation of the density changing with an elapsed time was confirmed to be within 5%, from which the structure was deemed to be sufficiently equilibrated.

Hereinafter, the functional film of the present invention, the method for forming the same, and the organic EL element will be described in detail. [1] Aromatic compound having polysubstituted structure

The functional film of the present invention is characterized by comprising an aromatic compound having a polysubstituted structure, having a condensed or noncondensed 6-membered aromatic hydrocarbon ring or aromatic heterocyclic ring having four or more condensed aromatic ring groups containing not less than 14π electrons, wherein three or more of the condensed aromatic ring groups containing not less than 14π electrons are adjacent to one another as substituents.

The aromatic compound having a polysubstituted structure according to the present invention is a compound in which a plurality of aromatic rings having not less than 14π electrons are bonded adjacent to a 6-membered aromatic ring. The present inventors have found, as a result of diligent experimentation, that the film condition can be maintained satisfactory not only upon film formation but also oiler an elapsed time by adjacently substituting the 6-membered ring with aromatic rings having a certain size or larger.

This is because a plurality of aromatic rings having not less than 14π electrons adjacent to one another on the mother nucleus of the 6-membered ring means that the sterically hindered groups cover the mother nucleus as substituents, forming the compound having the small surface area of the entire compound and a large steric hindrance. Such a compound not only inhibits the intramolecular movement but also inhibits the interaction with other molecules when subjected to an elevated temperature condition or an electric charge.

For example, JP 2006-100394A describes a compound in which a benzene ring is substituted adjacently with a plurality of aromatic rings having 10π electrons. However, such an aromatic ring having 10π electrons alone is not sufficient to inhibit intramolecular and intermolecular changes as described above under a long-term electric charging load and elevated temperature conditions with an elapsed time.

The aromatic compound having a polysubstituted structure according to the present invention is characterized in that in addition to the above structural features, the film containing only the compound according to the present invention, has a film density of a constant value calculated by molecular dynamics calculation, and the change in film density is small When the film is placed at elevated temperatures.

In the film containing only a certain compound as described above, the aforementioned intramolecular and intermolecular changes are in particular remarkable. The aromatic compound having a polysubstituted structure according to the present invention needs to have a density within a certain range even under such conditions, and a smaller change in film density at elevated temperatures. The present inventors have found, as a result of diligent experimentation, that the use of such a compound exhibits a remarkable effect on inhibiting changes with an elapsed time, especially in a film form, and a functional film that is stable even under an electric charging load or at elevated temperatures can be obtained.

The aforementioned JP 2006-100394A also discloses a compound in which a mother nucleus of a 6-membered ring is adjacently substituted with a plurality of aromatic rings having 14π electrons. Moreover, in recent years, use of a benzonitrile derivative (pentacarbazolylbenzonitrile: 2,3,4,5,6-pentakis(carbazo1-9-yl) benzonirile, abbreviated as “5CzBN” hereinafter) substituted with 5 carbazole ring groups as a light emitting material has been proposed, and research and development for practical use have been pursued. As described above, a compound having a structural feature, in which the mother nucleus of the 6-membered ring is adjacently substituted with a plurality of aromatic rings having not less than 14π electrons, has been known.

However, these were not contemplated to have intentionally such an effect as in the present invention, and even if the effect was accidentally expressed, the effect exhibited was insufficient for general industrial use. For example, JP 2006-100394A describes that a benzimidazole ring or an imidazopyridine ring is preferable as the nitrogen-containing aromatic heterocyclic ring, with which the mother nucleus of the 6-membered ring is substituted; however, the technical idea in the present invention was not disclosed in the literature. On the other hand, 5CzBN derivatives and these peripheral compounds that have been widely reported, are also light emitting materials used for doping a matrix, and they are not contemplated to have a favorable performance in a single film whereby remarkable intramolecular and intermolecular changes are produced.

As described above, although some of the compounds having structural characteristics according to the present invention are described in publicly known documents, the film density in a single film according to the present invention is not referred to, and the technical ideas found by the present inventors are not disclosed.

As a result, the use as a means of solving the problems was not easily guessed.

The condensed or noncondensed 6-membered aromatic hydrocarbon ring or aromatic heterocyclic ring that serves as the mother nucleus of the aromatic compound having a polysubstituted structure according to the present invention is preferably a noncondensed type. This is because the mother nucleus that is a noncondensed 6-aromatic ring is substituted with aromatic ring groups containing not less than 14π electrons that are adjacent to one another. As a result, not only the rotation of the single bond that bonds to the mother nucleus in the molecule can be inhibited but also the surface area of the entire compound can be minimized, which can inhibit the intermolecular interaction as well and further inhibit the fluctuation with an elapsed time. Moreover, a 6-membered aromatic hydrocarbon ring is preferable from the viewpoint of stability. Further, as described above, in order to further inhibit the fluctuation of the molecule, four or more condensed aromatic ring groups containing not less than 14π electrons are preferably adjacent to each other.

On the other hand, the number of condensed aromatic ring groups containing not less than 14π electrons that the aforementioned compound has is preferably five or less. These groups are advantageous for easier transfer due to their wide π-conjugated plane, on the contrary, are also susceptible to surrounding influences, where electric charges facilitate localization and interaction with each other in radical and excited states. In the present invention, not only the steric bulkiness is produced by the adjacent substitution, but also the five ring groups or less, allow the inhibition of intermolecular interaction of the highest occupied molecular orbitals (HOMO) and the lowest unoccupied molecular orbitals (LUMO) to be adjusted in an appropriate range and allow the interaction related to the deterioration of film quality to be inhibited while minimizing the deterioration of carrier transportability.

Moreover, the molecular weight of the aforementioned compound is preferably in the range of 900 to 2,000 and more preferably in the range of 1,000 to 2,000. This is because the large molecular weight can be expected to have an effect of increasing the decomposition temperature, and further, an increase in Tg leads to an improvement in stability with an elapsed time. However, in commonly used compounds, the higher the molecular weight, the greater the interaction and at the same time the vapor deposition temperature rises. For this reason, there arise the problems of being unable to carry out vapor deposition due to the decomposition at the vapor deposition temperature, and of lowering the reproducibility and of not allowing the material to be reused, due to the influence of decomposition products in the heating boat. The compound according to the present invention can inhibit an increase in the vapor deposition temperature owing to the characteristics thereof, even if the molecular weight is increased, and even if the molecular weight is 1,000 or more, both the stability improvement and vapor depositionability can be achieved. More preferably, it is in the range of 1,000 to 1,500.

The noncondensed 6-membered aromatic hydrocarbon ring represents a benzene ring and may be further substituted with a substituent.

The condensed 6-membered aromatic hydrocarbon ring includes, for example, a biphenylene ring, a naphthalene ring, an acenaphthene ring, a fluorene ring, a phenalene ring, an anthracene ring, a phenanthrene ring, a fluoranthrene ring, a pyrene ring, a chrysene ring, a triphenylene ring, a tetracene ring, a perylene ring, a pentacene ring, a pentaphene ring, a picene ring, a coronene ring, etc., and the naphthalene ring and the fluorene ring, the anthracene ring, the phenanthrene ring and the triphenylene ring are preferred, with the naphthalene ring and the fluorene ring being more preferred from the viewpoint of inhibiting crystallization. In particular the anthracene ring, phenanthrene ring and triphenylene ring are preferred from the viewpoint of improving the stability of the compound. These groups may be further substituted with a substituent, or they may be condensed with one another to further form a ring.

The aforementioned noncondensed 6-membered aromatic heterocyclic ring includes, for example, a pyridine ring, a pyridazine ring, a pyrimidine ring, a triazine ring, etc., and the pyridine ring and the pyrimidine ring are preferred, and in particular the pyridine ring is preferred. In addition, these groups may be further substituted with a substituent.

The condensed 6-membered aromatic heterocyclic ring includes, for example, an indole ring, a benzimidazole ring, a benzpyrazole ring, a benztriazole ring, an indolizine ring, a benzthiazole ring, a benzoxazole ring, a benzofuran ring, a benzothiophene ring, a quinoline ring, an isoquinoline ring, a cianoline ring, a quinazoline ring, a quinoxaline ring, a phthalazine ring, a naphthyridine ring,

a perimidine ring, a tepenidine ring, an acridine ring, a phenazine ring, a phenanthridine ring, a phenanthroline ring, a carbazole ring, a carboline ring, a diazacarbazole ring (representing any two or more carbon atoms constituting the carbazole ring, substituted with nitrogen atoms), a dibenzofuran ring, a dibenzothiophene ring, an azadibenzofuran ring, an azadibenzothiophene ring (representing any one of more carbon atoms constituting a benzothiophene ring or a dibenzofuran ring, substituted with nitrogen atoms), a phenoxathiin ring, a phenoxazine ring, a phenothiazine ring, a thiantolen ring, a naphthofuran ring, a naphthothiophene ring, an anthrafuran ring, an anthrathiophene ring, a quindrin ring, a quinindrin ring, an indoloindole ring, a benzofuroindole ring, a benzothiaindole ring, dibenzocarbazole ring, an indolocarbazole ring, an acrindoline ring, a triphenodithiazine ring, a triphenodioxazine ring, a phenanthrazine ring, etc., and preferably a ring containing at least one N atom, and more preferably the indole ring, benzimidazole ring, indolizine ring, quinolone ring, isoquinoline ring, quinazoline ring, acridine ring, phenazine ring, phenanthroline ring, carbazole ring, carboline ring, azadibenzofuran ring, and azadibenzothiophene ring. From the viewpoint of electron transportability, the benzimidazole ring, phenanthroline ring, azadibenzofuran ring or azadibenzothiophene ring is particularly preferred, and from the viewpoint of hole transportability, the indole ring, carbazole ring, carboline ring and indolizine ring are preferred. These groups may be further substituted with a substituent, or they may be condensed with one another to further form a ring.

The condensed aromatic ring group comprising 14π electrons represents a condensed aromatic hydrocarbon ring or a condensed aromatic heterocyclic ring containing not less than 14π electrons, and is preferably a condensed aromatic heterocyclic ring from the viewpoint of charge transportability.

The condensed aromatic hydrocarbon ring containing not less than 14π electrons includes, for example, an anthracene ring, a phenanthrene ring, a fluoranthrene ring, a pyrene ring, a chrysene ring, a triphenylene ring, a tetracene ring, a perylene ring, a pentacene ring, a pentaphene ring, a picene ring, a coronene ring, etc., and it is preferably a condensed aromatic ring group of not less than 14π electrons, and more preferably the anthracene ring and the phenanthrene ring. Moreover, the group preferably does not have a plane of symmetry. These groups may be further substituted with a substituent, or they may be condensed with one another to further form a ring.

The condensed aromatic heterocyclic ring containing not less than 14π electrons includes, for example, a perimidine ring, a tepenidine ring, an acridine ring, a phenazine ring, a phenanthridine ring, a phenanthroline ring, a carbazole ring, a carboline ring, a diazacarbazole ring, a dibenzofuran ring, a dibenzothiophene ring, an azadibenzofuran ring, an azadibenzothiophene ring, a phenoxathiin ring, a phenoxazine ring, a phenothiazine ring, a thiantolen ring, a naphthofuran ring, a naphthothiophene ring, an anthrafuran ring, an anthrathiophene ring, a quindrin ring, a quinindrin ring, an indoloindole ring, a benzofuroindole ring, abenzothiaindole ring, a dibenzocarbazole ring, an indolocarbazole ring, an acrindoline ring, a triphenodithiazine ring, a triphenodioxazine ring, a phenanthrazine ring, etc., and it is preferably a group having at least one N atom, and more preferably a group having two or more N atoms. Further, it is preferably a group containing not less than 14π electrons, and more preferably the carbazole ring, the carboline ring, the diazacarbazole ring, the dibenzothran ring, the dibenzothiophene ring, the azadibenzofuran ring, and the azadibenzothiophene ring. Moreover, it is preferably a group having no plane of symmetry, and more preferably the carboline ring, the azadibenzofuran ring, and the azadibenzothiophene ring, and particularly preferably the carboline ring. Among them, δ-carboline is preferable from the viewpoint of electron transportability, and α-carboline is preferable from the viewpoint of stability. These groups may be further substituted with a substituent, or they may be condensed with one another to further form a ring.

The aforementioned compound preferably has one or more chirality generation sites. By having the chirality generation site in the molecule, it becomes a molecule containing optical isomers. Since the optical isomers have the same structural formula with one another, the basic physical characteristics are almost the same, and even if a functional film is fabricated from a compound containing a plurality of optical isomers, the physicochemical properties of the film are almost unchanged. Since the optical isomers have different steric configurations from one another, on the other hand, the intermolecular interaction is inhibited, which can not only prevent crystallization upon forming of the functional film, but also inhibit changes in film quality with an elapsed time, such as upon drive and storage at elevated temperatures. From this, one or more chirality generation sites allow the effect of maintaining the film stability with further elapsed time to be expected, which leads to long service life and resistance to drive voltage fluctuation, and enable to inhibit the vapor deposition temperature rise due to increase in molecular weight, and further to minimize occurrence of burning of the vapor deposition boat.

Further, in the present invention, the number of chirality generation sites is preferably two or more and more preferably four to five. This is because an enantiomer and a diastereomer are present in an optical isomer, but the diastereomer is formed when two or more chirality generation sites are present, and therefore, the presence of two or more chirality generation sites include a diastereomer in addition to the enantiomer, allowing the effect of inhibiting the intermolecular interaction to be expected. Further, in the present invention, among three or more condensed aromatic ring groups having not less than 14π electrons which are adjacent to one another as substituents, the substituent in the middle is preferably a group having no plane of symmetry and particularly preferably a compound having no plane of symmetry. This is because a compound having a plane of symmetry, even with a chirality generation site, becomes optically inactive, and the number of optical isomers will be decreased.

In the present invention, the aromatic compound having the aforementioned polysubstituted structure preferably has the structure represented by the following general formula (1).

[Chemical Formula 3]

Ar [symbol] (HetAr)ₙ General formula (1)

wherein Ar represents substituted or unsubstituted heteroaryl, or substituted or unsubstituted aryl; HetAr represents a substituted or unsubstituted condensed aromatic heterocyclic group containing not less than 14π electrons; and n represents an integer of 4 or more.

In the general formula (1), the substituted or unsubstituted heteroaryl represents a group similar to the aforementioned condensed or noncondensed 6-membered aromatic heterocyclic ring.

The substituted or unsubstituted aryl represents a group similar to the condensed or noncondensed 6-membered aromatic hydrocarbon ring.

HetAr represents substituted or unsubstituted condensed aromatic heterocyclic groups containing not less than 14π electrons, wherein three or more of the HetAr are bonded to Ar adjacent to one another as substituents.

The subscript n represents an integer of 4 or more, but is particularly preferably 5 or less.

When n is 4, preferably all four HetAr are adjacently bonded to Ar, and when n is 5 or more, preferably at least all of five HetAr are adjacently bonded to Ar.

The compound having the structure represented by the general formula (1) preferably has the structure represented by the general formula (2).

[Chemical Formula 4]

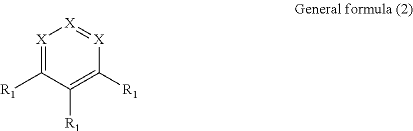

wherein X represents N or CR₂ and at least one X represents CR₂; R₁ represents a condensed aromatic heterocyclic group containing not less than 14π electrons; R₂ represents a hydrogen atom or R₁, or represents any group selected from among an alkyl group, a cycloalkyl group, an alkenyl group, an aromatic hydrocarbon group, an aromatic heterocyclic group, a heterocyclic group, an alkoxy group, a cycloalkoxy group, an aryloxy group, an alkylthio group, a cycloalkylthio group, an arylthio group, an aryloxycarbonyl group, a sulfamoyl group, an alkoxycarbonyl group, an acyl group, an acyloxy group, an amide group, a carbamoyl group, a ureido group, a sulfinyl group, an alkylsulfonyl group, an arylsulfonyl group or a heteroalylsulfonyl group, an amino group, a halogen atom, a fluorinated hydrocarbon group, a cyano group, a nitro group, a hydroxy group, a mercapto group, a silyl group, and a phosphono group; Here, at least one R₂ represents R₁.

In the general formula (2), X represents N or CR₂, and at least one X represents CR₂.

R₁ represents a condensed aromatic heterocyclic group containing not less than 14π electrons.

R₂ represents a hydrogen atom or R₁, or includes alkyl groups (for example, a methyl group, an ethyl group, a propyl group, an isopropyl group, a tert-butyl group, a pentyl group, a hexyl group, an octyl group, a dodecyl group, a tridecyl group, a tetradecyl group, a pentadecyl group, etc.), cycloalkyl groups (for example, a cyclopentyl group, a cyclohexyl group, etc.), alkenyl groups (for example, a vinyl group, an allyl group, etc.), alkynyl groups (for example, an ethynyl group, a propargyl group, etc.), aromatic hydrocarbon groups (for example, a phenyl group, a p-chlorophenyl group, a mesityl group, a tolyl group, a xylyl group, a naphthyl group, an anthryl group, a azulenyl group, an acenaphthenyl group, a fluorenyl group, a phenanthryl group, an indenyl group, a pyrenyl group, a biphenylyl group, etc.), aromatic heterocyclic groups (for example, a pyridyl group, a pyrimidinyl group, a furyl group, a pyrrolyl group, an imidazolyl group, a benzimidazolyl group, a pyrazolyl group, a pyrazinyl group, triazolyl groups (for example, 1,2,4-triazol-1-yl group, 1,2,3-triazol-1-yl group, etc.), a pyrazolotriazolyl group, an oxazolyl group, a benzoxazolyl group, a thiazolyl group, an isoxazolyl group, an isothiazolyl group, a frazayl group, a thienyl group, a quinolyl group, a benzolinyl group, a dibenzofuryl group, a benzothienyl group, a dibenzothienyl group, an indolyl group, a carbazolyl group, a carbolinyl group, a diazacarbazolyl group (one of the carbon atoms constituting the carboline ring of the carbolinyl group is substituted with a nitrogen atom), a quinoxalinyl group, a pyridazinyl group, a triazinyl group, a quinazolinyl group, a phthalazinyl group, etc.), heterocyclic groups (for example, a pyrrolidyl group, an imidazolidyl group, a morpholic group, an oxazolidyl group, etc.), alkoxy groups (for example, a methoxy group, an ethoxy group, a propyloxy group, a pentyloxy group, a hexyloxy group, an octyloxy group, a dodecyloxy group, etc.), cycloalkoxy groups (for example, a cyclopentyloxy group, a cyclohexyloxy group, etc.), aryloxy groups (for example, a phenoxy group, a naftyloxy group, etc.), alkylthio groups (for example, a methylthio group, an ethylthio group, a propylthio group, a pentylthio group, a hexylthio group, an octylthio group, a dodecylthio group, etc.), cycloalkylthio groups (for example, a cyclopentylthio group, a cyclohexylthio group, etc.), arylthio groups (for example, a phenylthio group, a naphylthio group, etc.), alkoxycarbonyl groups (for example, a methyloxycarbonyl group, an ethyloxycarbonyl group, a butyloxycarbonyl group, an octyloxycarbonyl group, a dodecyloycarbonyl group, etc.), myloxycarbonyl groups (for example, a phenyloxycarbonyl group, a naphthyloxycarbonyl group, etc.), sulfamoyl groups (for example, an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, a 2-pyridylaminosulfonyl group, etc.), acyl groups (for example, an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, a pyridylcarbonyl group, etc.), acyloxy groups (for example, an acetyloxy group, an ethylcarbonyloxy group, a butylcarbonyloxy group, an octylcarbonyloxy group, a dodecylcarbonyloxy group, a phenylcarbonyloxy group, etc.), amid groups (for example, a methylcarbonylamino group, an ethylcarbonylamino group, a dimethylcarbonylamino group, a propylcarbonylamino group, a pentylcarbonylamino group, a cyclohexylcarbonylamino group, a 2-ethythexylcarbonylamino group, an octylcarbonylamino group, a dodecylcarbonylamino group, a phenylcarbonylamino group, a naphthylcarbonylamino group, etc.), carbamoyl groups (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, dodecylaminocarbonyl group, a phenylaminocarbonyl group, a naphthylaminocarbonyl group, a 2-pyridylaminocarbonyl group, etc.), ureido groups (for example, a methyl ureido group, an ethyl ureido group, a pentyl ureido group, a cyclohexyl ureido group, an octyl ureido group, a dodecyl ureido group, a phenyl ureido group, a naphthyl ureido group, a 2-pyridyl amino ureido group, etc.), sulfinyl groups (for example, a methylsulfinyl group, an ethylsulfinyl group, a butylsulfinyl group, a cyclohexylsulfinyl group, a 2 -ethylhexylsulfinyl group, a dodecylsulfinyl group, a phenylsulfinyl group, a naphthylsulfinyl group, a 2-pyridylsulfinyl group, etc.), alkylsulfonyl groups (for example, a methylsulfonyl group, an ethylsulfonyl group, a butylsulfonyl group, a cyclohexylsulfonyl group, a 2-ethythexylsulfonyl group, a dodecylsulfonyl group, etc.), arylsulfonyl groups or heteroarylsulfonyl groups (for example, a phenylsulfonyl group, a naphthylsulfonyl group, a 2-pyridylsulfonyl group, etc.), amino groups (for example, an amino group, an ethylamino group, a dimethylamino group, a diphenylamino group, a diisopropylamino group, a ditert-butyl group, a cyclohexylamino group, a butylamino group, a cyclopentylamino group, a 2-ethylhexylamino group, a dodecylamino group, an anilino group, a naphthylamino group, a 2-pyridylamino group, etc.), halogen atoms (for example, a fluorine atom, a chlorine atom, a bromine atom, etc.), fluorinated hydrocarbon groups (for example, a fluoromethyl group, a trifluoromethyl group, a pentafluoroethyl group, a pentafluorophenyl group, etc.), a cyano group, a nitro group, a hydroxy group, a mercapto group, silyl groups for example, a trimethylsilyl group, a triisopropylsilyl group, a triphenylsilyl group, a phenyldiethylsilyl group, etc.), or a phosphono group, etc. These groups may be further substituted with a substituent, or they may be condensed with one another to further form a ring.

R₂ is preferably a group other than hydrogen, and having an appropriate steric hindrance is expected to have an effect of improving stability with an elapsed time, and therefore, it represents R₁, or represents preferably any group selected from among a cycloalkyl group, an aromatic hydrocarbon group, an aromatic heterocyclic group, a heterocyclic group, an cycloalkoxy group, an aryloxy group, a cycloalkylthio group, an arylthio group, an amino group, a fluorinated hydrocarbon group, a nitro group, a silyl group, and a phosphono group. In particular, it represents R₁ or represents preferably any group selected from among a cycloalkyl group, an aromatic hydrocarbon group, an aromatic heterocyclic group, an amino group, a fluorinated hydrocarbon group, a nitro group, a silyl group, and a phosphono group.

One R₂ represents R₁, and two R₂ are each particularly preferably R₁.

When R₂ does not represent R₁, it is preferably any group selected from among a cycloalkyl group, an aromatic hydrocarbon ring, a fluorinated hydrocarbon group, and a silyl group from the viewpoint of improving compound stability. When it is an aromatic hydrocarbon ring group, it preferably further has a substituent. The substituent is preferably any group selected from among an alkyl group, a cycloalkyl group, an aromatic hydrocarbon group, an aromatic heterocyclic group, a heterocyclic group, an amino group, a halogen atom, a fluorinated hydrocarbon group, a cyano group, a nitro group, a silyl group and a phosphono group.

Moreover, when R₂ does not represent it may be any group selected from among an aromatic heterocyclic ring, an aromatic heterocyclic group, a heterocyclic group, an amino group, or a phosphono group from the viewpoint of charge transportability. In particular, it preferably contains one or more nitrogen atoms.

The functional film of the present invention preferably contains 50% by weight or more of the aromatic compound according to the present invention, more preferably 75% by weight or more, and still more preferably 96% by weight or more, and it is particularly preferably a film comprising only the aromatic compound according to the present invention. This is because the aromatic compound according to the present invention is particularly effective in a situation where the influence of the interaction is remarkably exhibited.

Hereinafter, compounds having the poly substituted structure according to the present invention will be exemplified, but the present invention is not limited thereto.

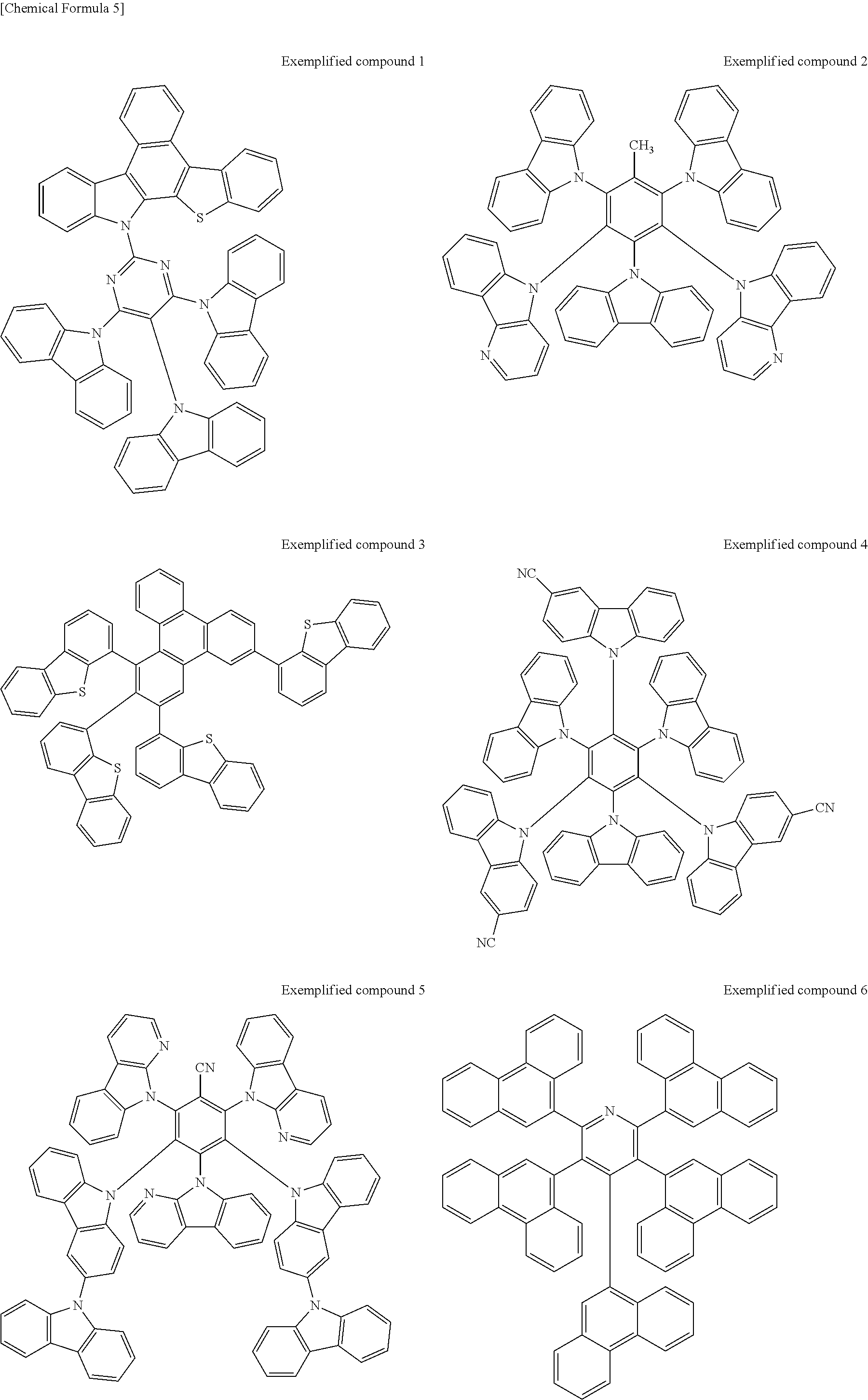

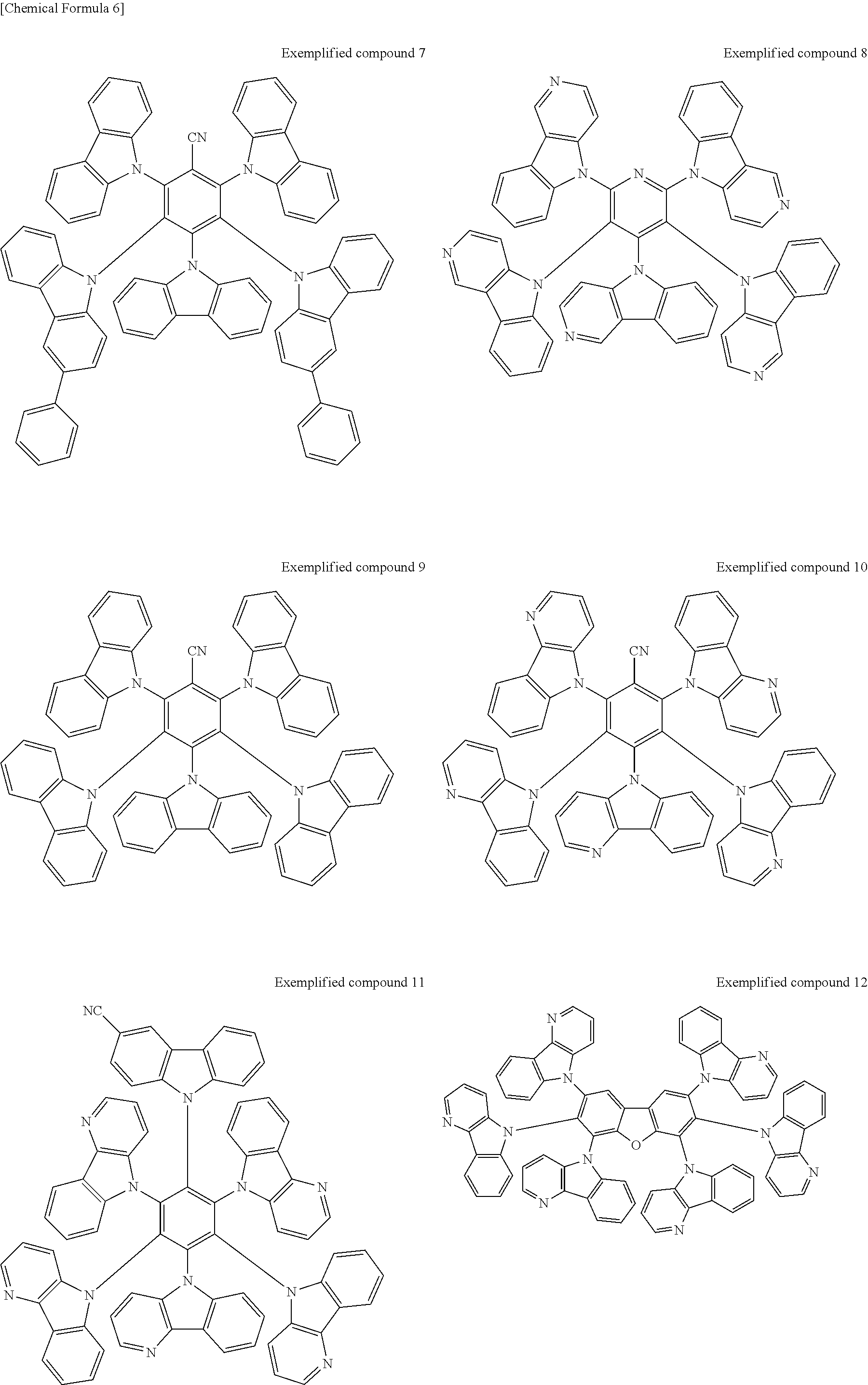

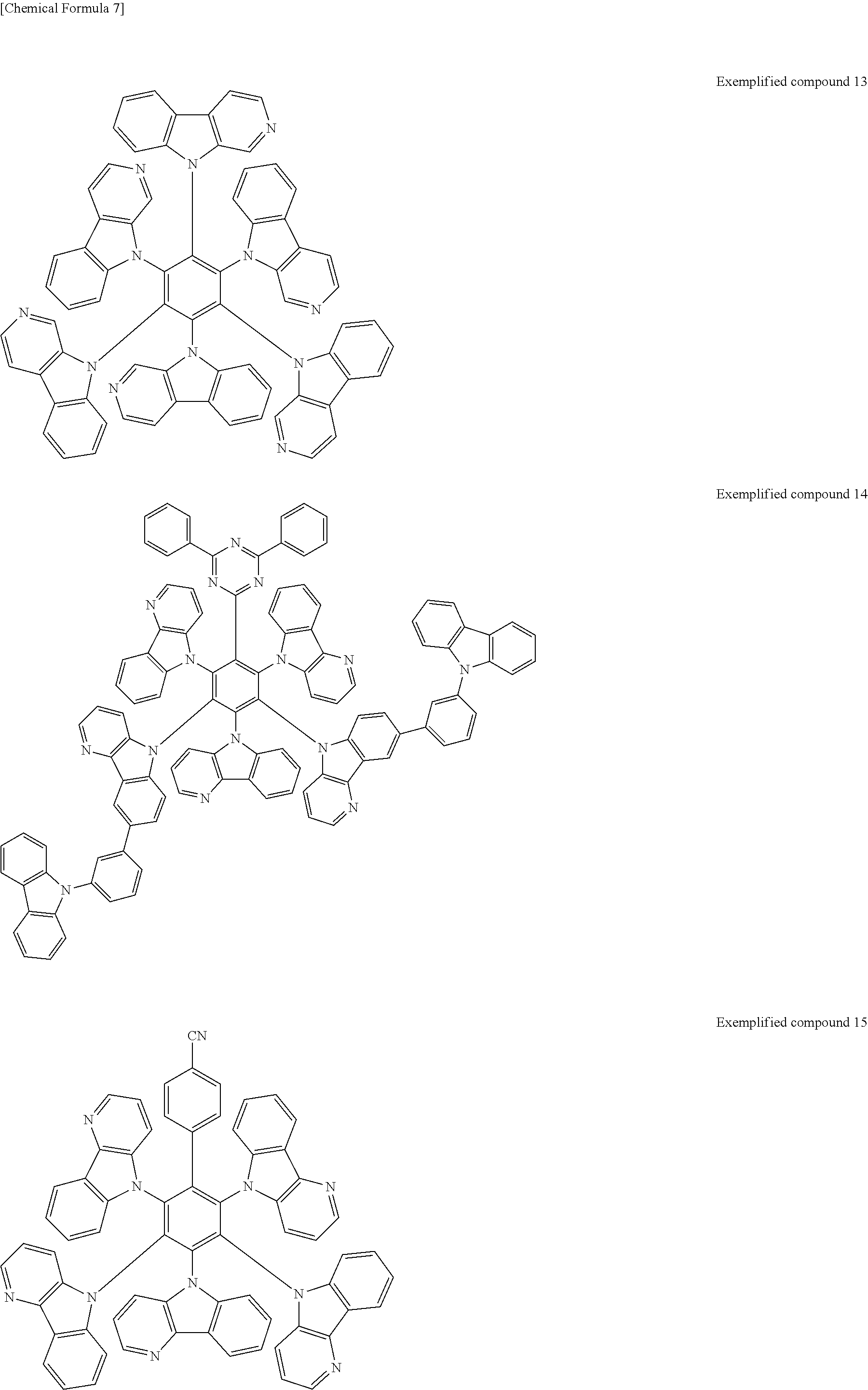 [SMILES: c1ccc2c(c1)c1ccncc1n2-c1c(-n2c3ccccc3c3ccncc32)c(-n2c3ccccc3c3ccncc32)c(-n2c3ccccc3c3ccncc32)c(-n2c3ccccc3c3ccncc32)c1-n1c2ccccc2c2ccncc21 ; c1ccc(-c2nc(-c3ccccc3)nc(-c3c(-n4c5ccccc5c5ncccc54)c(-n4c5ccc(-c6cccc(-n7c8ccccc8c8ccccc87)c6)cc5c5ncccc54)c(-n4c5ccccc5c5ncccc54)c(-n4c5ccc(-c6cccc(-n7c8ccccc8c8ccccc87)c6)cc5c5ncccc54)c3-n3c4ccccc4c4ncccc43)n2)cc1 ; N#Cc1ccc(-c2c(-n3c4ccccc4c4ncccc43)c(-n3c4ccccc4c4ncccc43)c(-n3c4ccccc4c4ncccc43)c(-n3c4ccccc4c4ncccc43)c2-n2c3ccccc3c3ncccc32)cc1]

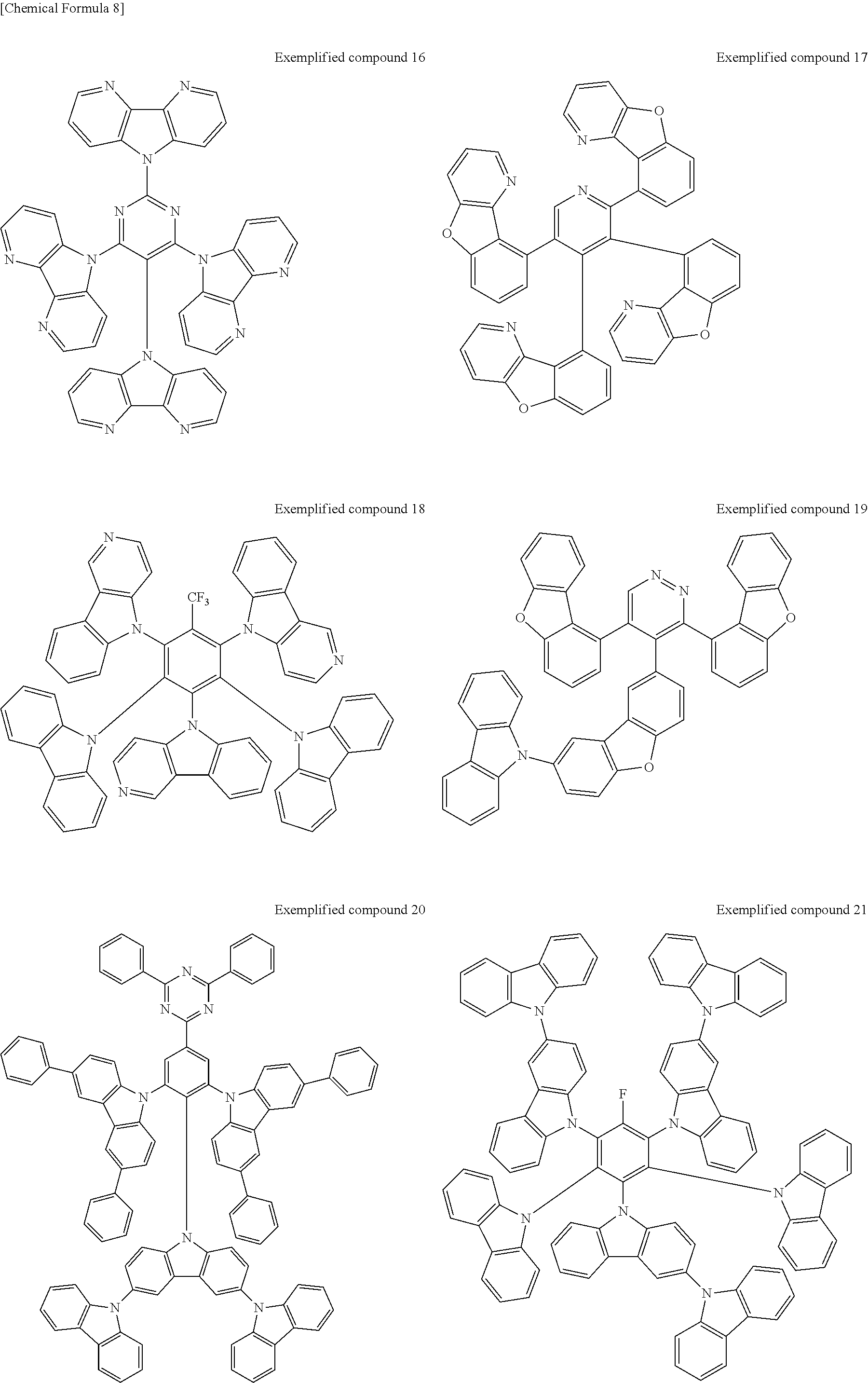

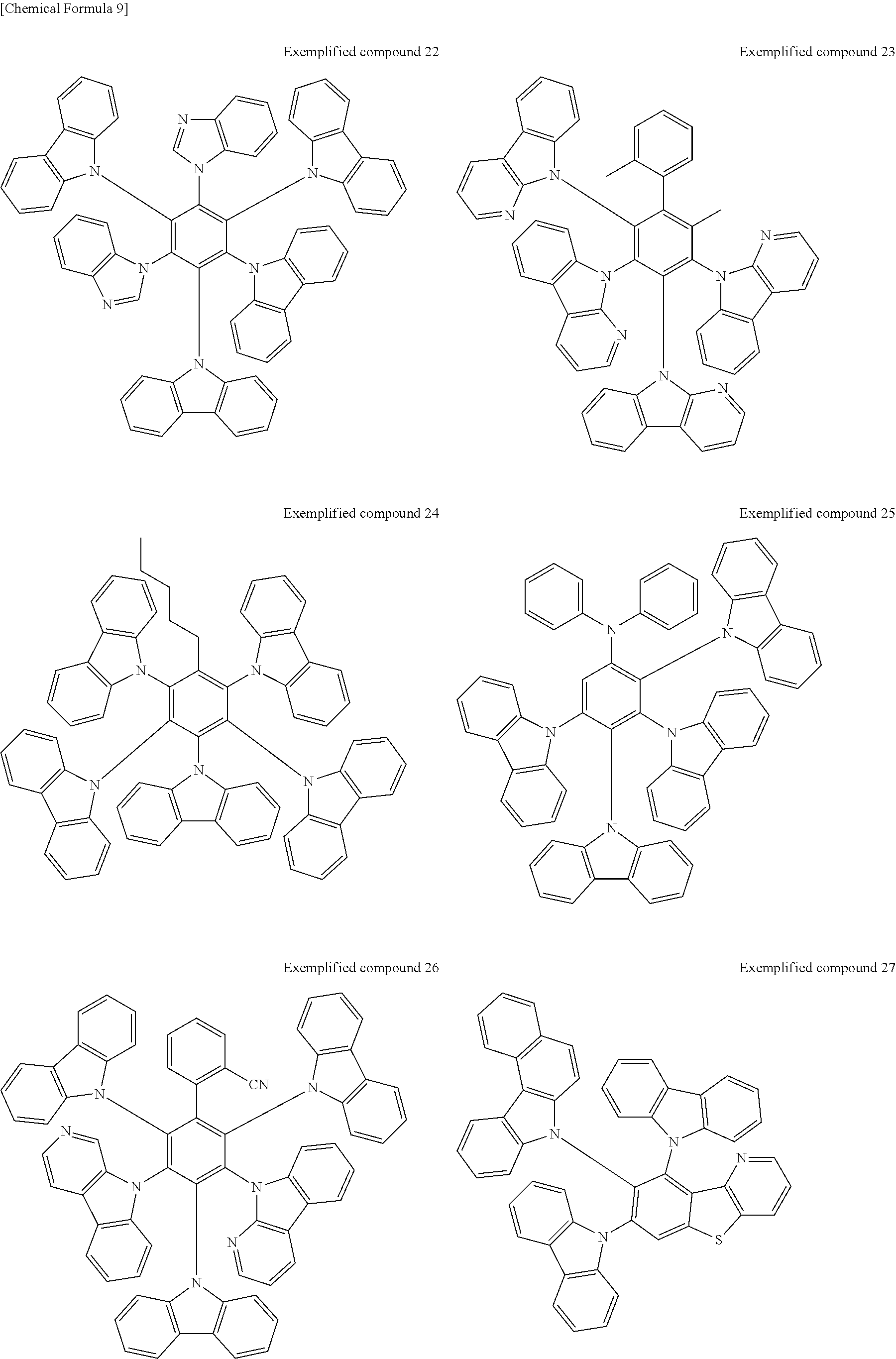

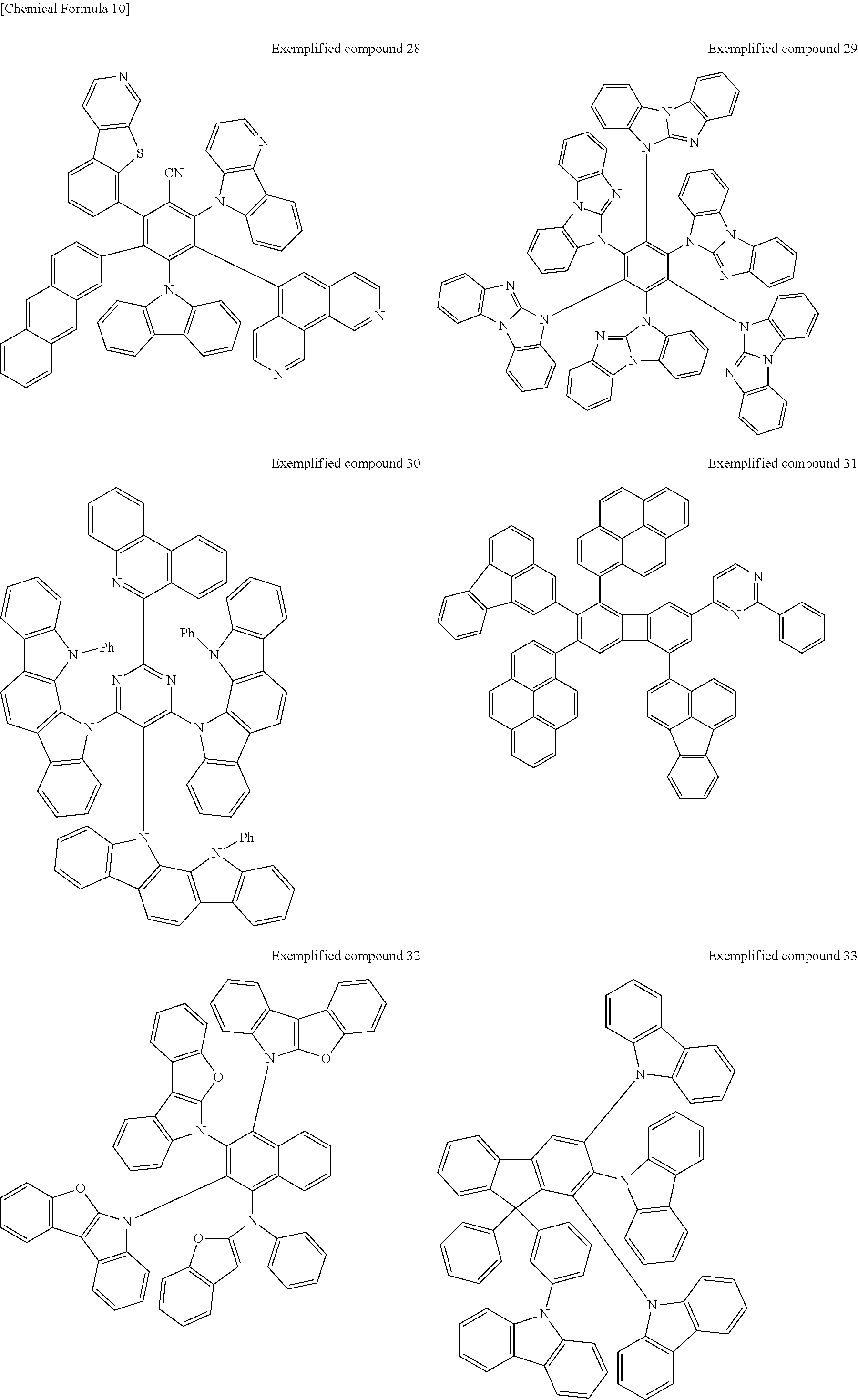

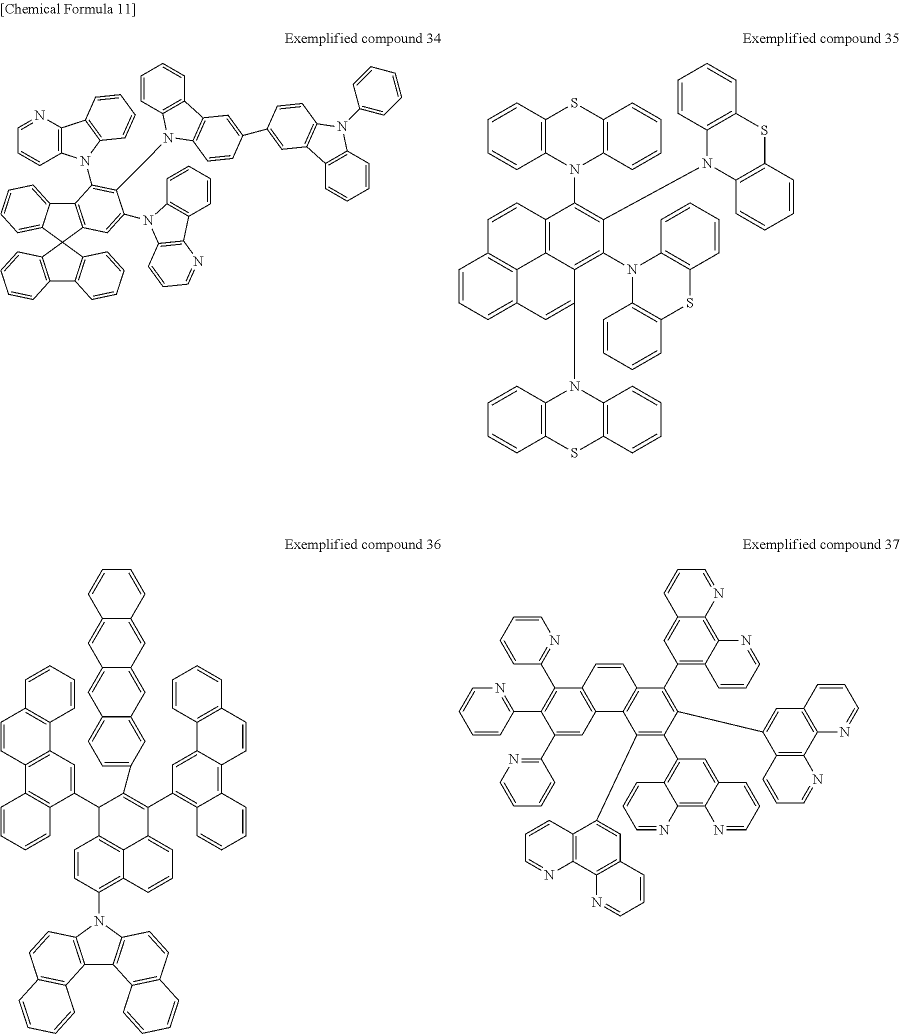

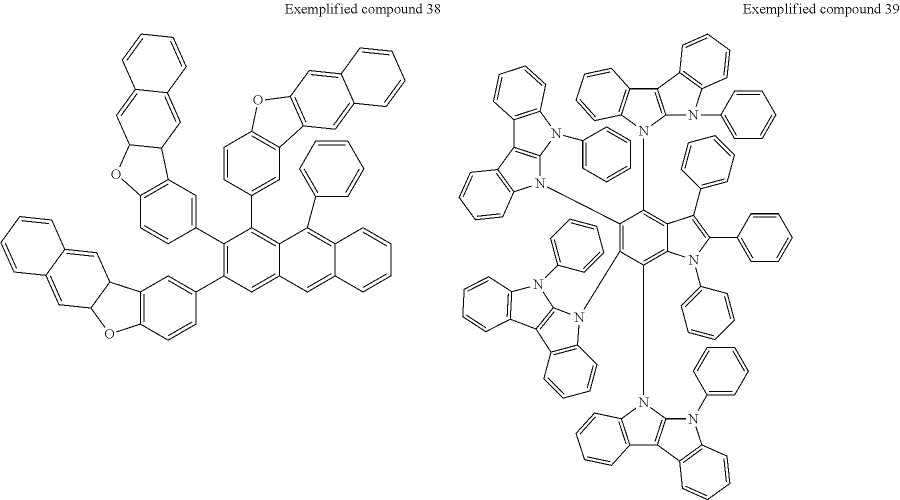 [SMILES: C1=c2ccccc2=CC2c3cc(-c4cc5cc6ccccc6c(-c6ccccc6)c5c(-c5ccc6oc7cc8ccccc8cc7c6c5)c4-c4ccc5c(c4)C4C=c6ccccc6=CC4O5)ccc3OC12 ; c1ccc(-c2c(-c3ccccc3)n(-c3ccccc3)c3c(-n4c5ccccc5c5c6ccccc6n(-c6ccccc6)c54)c(-n4c5ccccc5c5c6ccccc6n(-c6ccccc6)c54)c(-n4c5ccccc5c5c6ccccc6n(-c6ccccc6)c54)c(-n4c5ccccc5c5c6ccccc6n(-c6ccccc6)c54)c23)cc1]

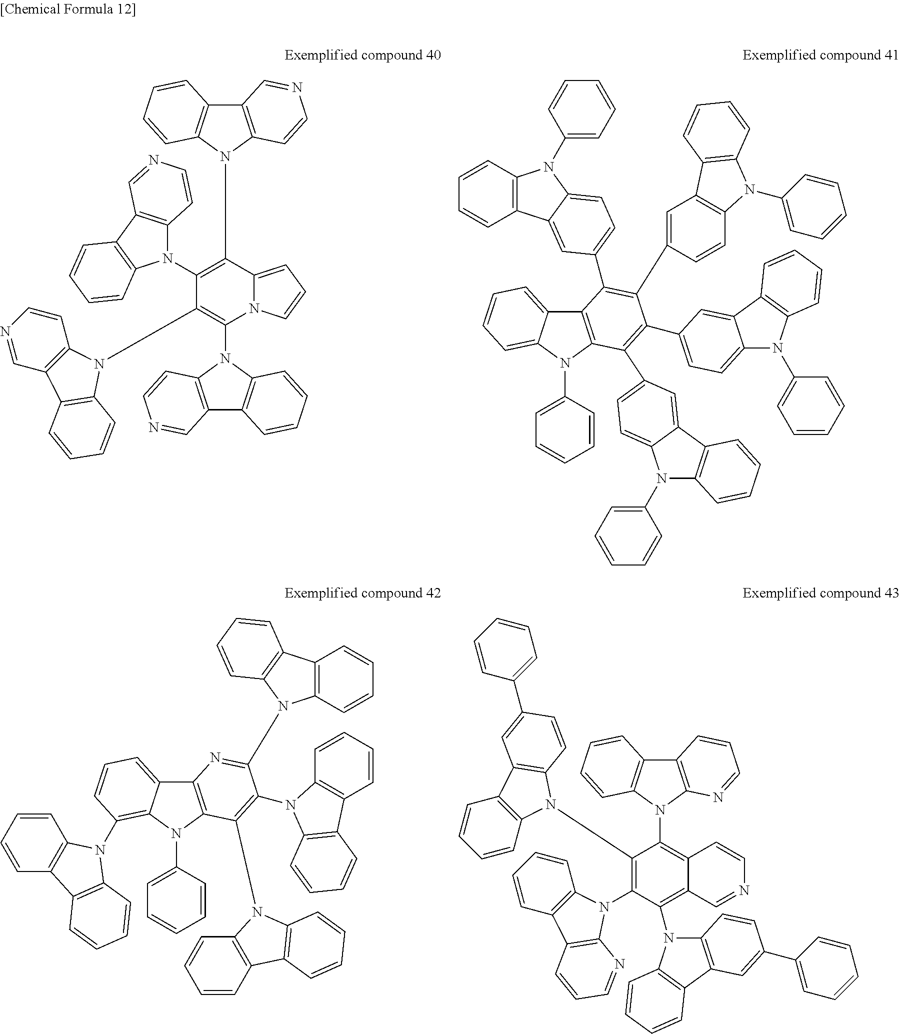

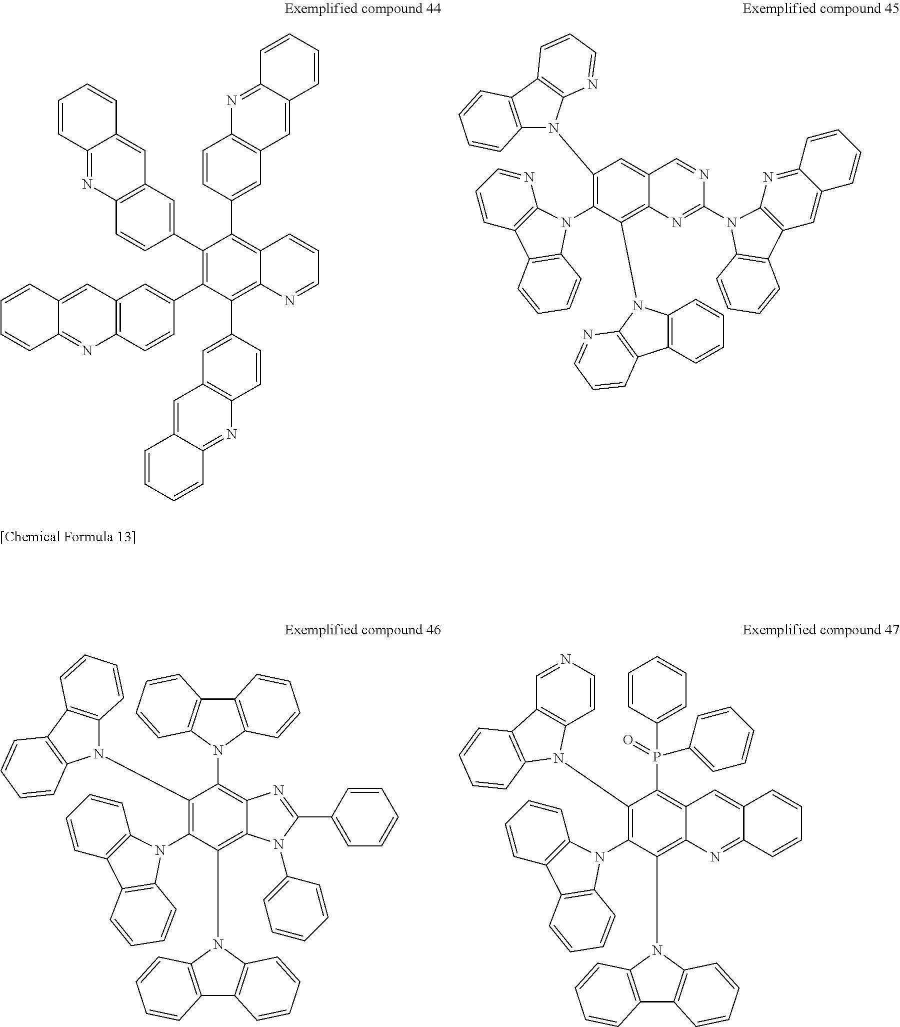 [SMILES: c1ccc2nc3ccc(-c4c(-c5ccc6nc7ccccc7cc6c5)c(-c5ccc6nc7ccccc7cc6c5)c5ncccc5c4-c4ccc5nc6ccccc6cc5c4)cc3cc2c1 ; c1ccc2nc3c(cc2c1)c1ccccc1n3-c1ncc2cc(-n3c4ccccc4c4cccnc43)c(-n3c4ccccc4c4cccnc43)c(-n3c4ccccc4c4cccnc43)c2n1 ; c1ccc(-c2nc3c(-n4c5ccccc5c5ccccc54)c(-n4c5ccccc5c5ccccc54)c(-n4c5ccccc5c5ccccc54)c(-n4c5ccccc5c5ccccc54)c3n2-c2ccccc2)cc1 ; O=P(c1ccccc1)(c1ccccc1)c1c(-n2c3ccccc3c3cnccc32)c(-n2c3ccccc3c3ccccc32)c(-n2c3ccccc3c3ccccc32)c2nc3ccccc3cc12]

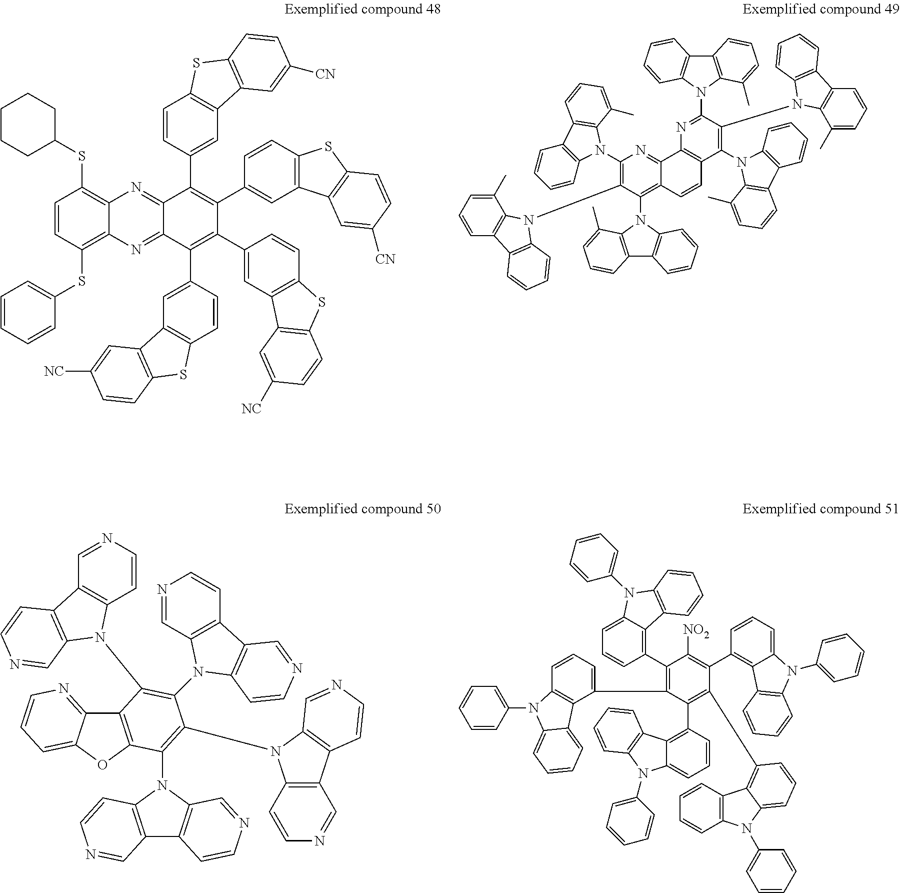

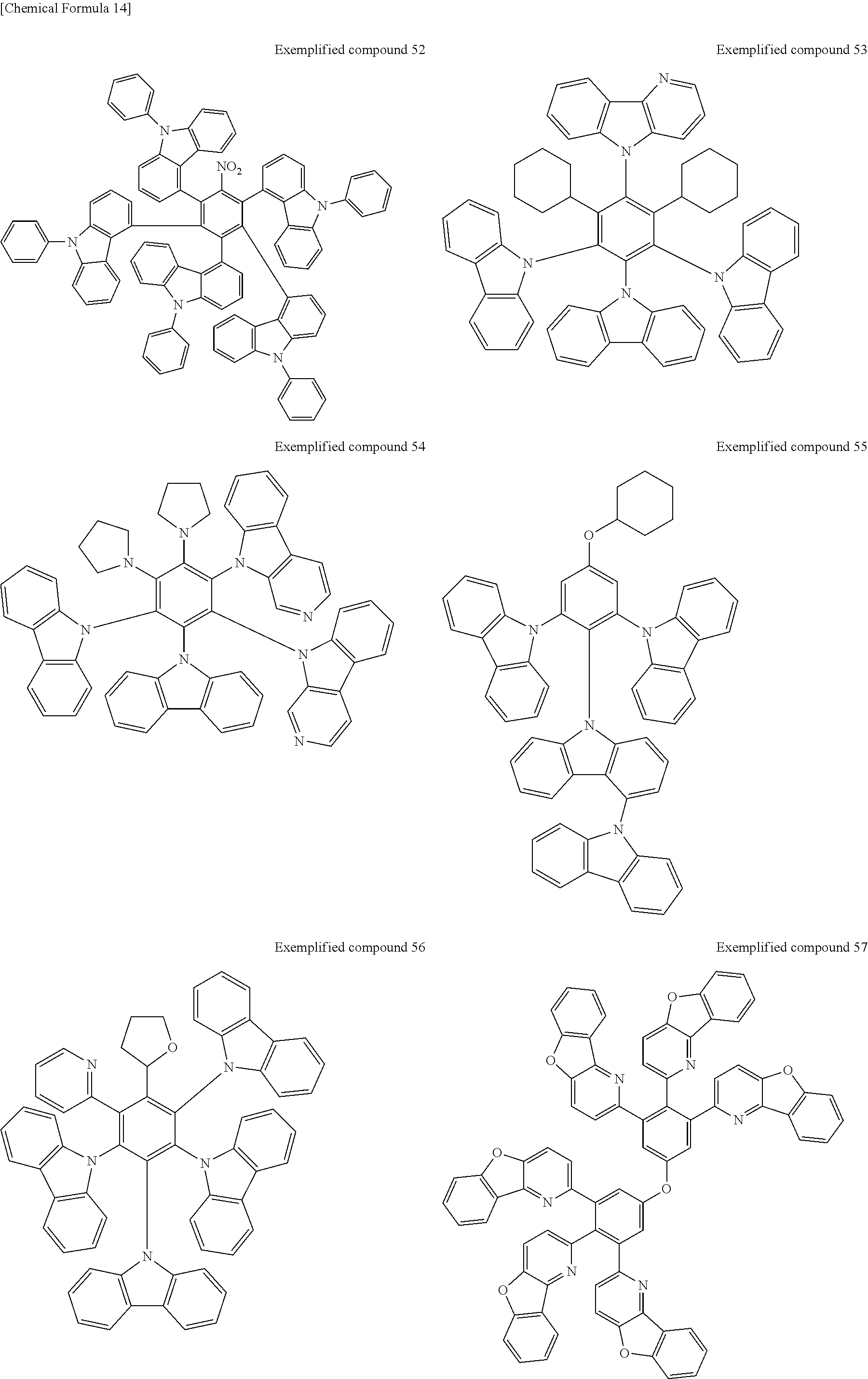

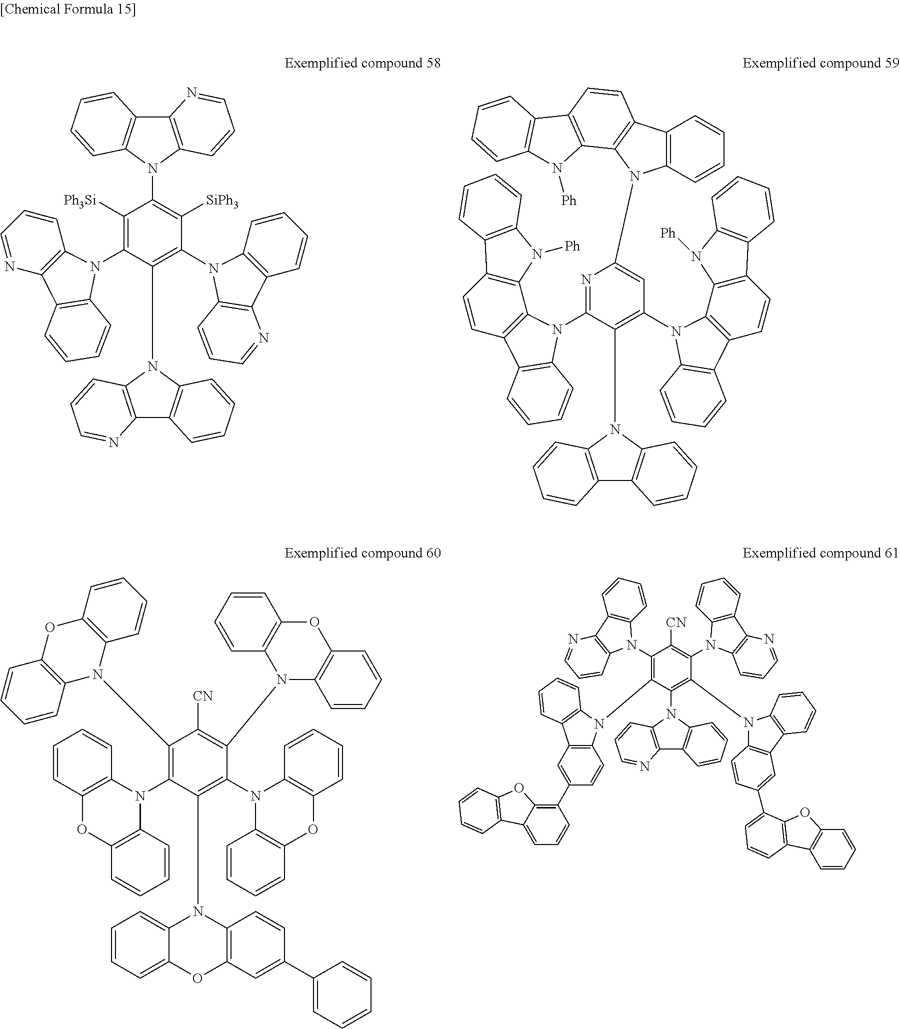

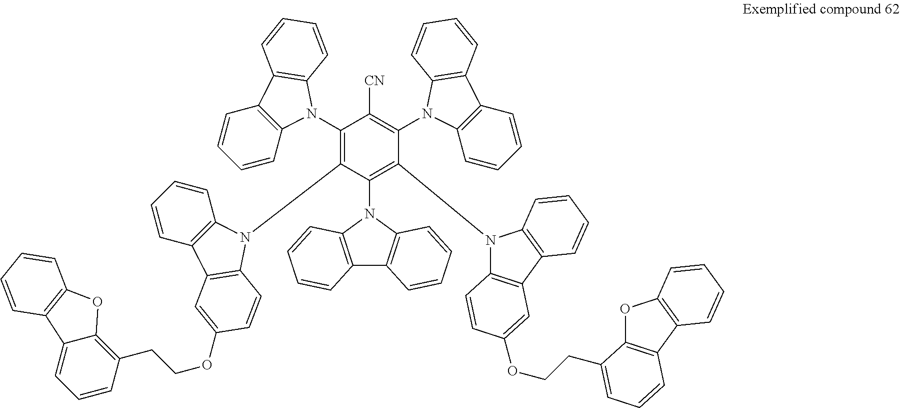 [SMILES: N#Cc1c(-n2c3ccccc3c3ccccc32)c(-n2c3ccccc3c3cc(OCCc4cccc5c4oc4ccccc45)ccc32)c(-n2c3ccccc3c3ccccc32)c(-n2c3ccccc3c3cc(OCCc4cccc5c4oc4ccccc45)ccc32)c1-n1c2ccccc2c2ccccc21]

[2] Method for Forming, Functional Film

When in the functional film of the present invention, the film density value calculated by the molecular dynamics calculation of NPT ensemble at 300 K for the compound having the polysubstituted structure is an initial film density of the functional film containing only the compound, and when the film density value calculated by the molecular dynamics calculation at 370 K for the aromatic compound is the film density value after storage of the functional film stored under the temperature, the difference between the initial film density and the film density value after storage is 1% or less with respect to the initial film density.

Controlling the initial film density value within the range and the difference between the initial film density and the film density value after storage within the above value range can be achieved by appropriately selecting the structure of the compound used in the film formation of the functional film.

A method for forming the functional film of the present invention includes methods, such as a spin coating method, a casting method, an inkjet method, a vapor deposition method, a printing method, etc., but is preferably a method such as the spin coating method or the vapor deposition method from the viewpoint of facilitating to obtain a uniform film and of hardly forming pinholes. In particular the vapor deposition method is preferably employed for forming a film having the initial film density within the range according to the present invention or for forming a film in which fluctuations in film density before and after storage are controlled.

Physical vapor deposition methods, such as a vacuum vapor deposition method, ion beam deposition method, ion plating method, etc., for vapor deposition systems, and a sputtering method, an ion beam sputtering method, a magnetron sputtering method, etc., for sputtering systems, are known, and further, a film can also be formed by chemical vapor deposition methods, such as a thermal CVD method, a catalytic chemical vapor deposition method (Cat-CVD), a capacitively coupled plasma CVD method (CCP-CVD), an optical CVD method, a plasma CVD method (PE-CVD), an epitaxial growth method, ash atomic layer growth method, etc. The vacuum vapor deposition method, ion beam deposition method, ion plating method, or sputtering method are preferably employed in the present invention.

When the vacuum vapor deposition method is adopted for film formation, the vapor deposition conditions vary depending on the type of compound used, etc., but in general preferably appropriately selected from the boat heating temperature within the range of 50 to 450° C., the degree of vacuum within the range of 1×10⁻⁶ to 1×10⁻² Pa, the vapor deposition rate within the range of 0.01 to 50 nm/sec, and the substrate temperature within the range of −50 to 300° C.

The method for forming the functional film of the present invention is preferably a method for consistently forming a film by the vacuum vapor deposition method in a single vacuum, but may be a different method for forming a film, for example, by taking out a film in the middle followed by forming with a wet process. The working environment in this case is preferably carried out in a dry inert gas atmosphere.

The vacuum vapor deposition method includes, for example, resistance heating vapor deposition, high frequency induction heating vapor deposition, electron beam deposition, ion beam deposition, plasma-assisted vapor deposition, etc. The vacuum vapor deposition method is a method in which a material to be formed into a film is evaporated or sublimated in a vacuum, and the vapor reaches a substrate (an object or a place to which the film is to be attached) and is deposited to form a film. Since the vaporized material reaches the substrate as it is without being electrically applied-to the evaporation material or the substrate, a highly pure film with less damage to the substrate can be formed. An example of the vacuum vapor deposition method preferably includes a vacuum vapor deposition method by using an ion assisted deposition (IAD) method. The IAD method is a method in which the high kinetic energy of ions is applied during film formation to form a dense film, or the adhesion of the film is enhanced. For example, the method by using an ion beam is a method for accelerating an adherend material with ionized gas molecules irradiated from an ion source to form a film on the substrate surface. The IAD method is also referred to as an “ion beam assist method”. The details are described JP 2003-221663A, WO2015/030015, etc.

The sputtering method includes, for example, reactive sputtering methods such as magnetron cathode sputtering, flat plate magnetron sputtering, 2-pole AC flat plate magnetron sputtering, and 2-pole AC rotation magnetron sputtering. The sputtering method is a method for colliding particles having high energy such as plasma with a material (target), striking out the material components by the impact, and depositing the particles on a substrate to form a film. The alloy components can be deposited on the substrate almost as they are because the materials themselves are stricken out.

The ion plating method includes a DC ion plating method, RF ion plating method, etc. The ion plating method has almost the same principle as the vapor deposition method, except that the vaporized particles are allowed to pass through the plasma to be positively charged, and a negative charge is applied to the substrate to attract and deposit the vaporized particles followed by forming a film, which allows forming of a film having stronger adhesion with the lower layer than that of the vapor deposition method.

The thickness of the functional film of the present invention (the total thickness when a plurality of layers are laminated) is preferably about 5 nm to 5 μm, preferably about 5 to 200 nm. If the thickness is 5 nm or more, a functional layer having low voltage drive, high luminous efficiency, long se vice life and resistance to chive voltage fluctuation, is obtained, and the thickness of 5 μm or less can prevent the surface of the multilayer film from surface deformation due to the film stress itself.

### [3] Organic EL Element

The organic EL element of the present invention is an organic electroluminescence element comprising at least a pair of electrodes and one or a plurality of layers, wherein at least one of the layers has the functional film of the present invention. In particular, the functional film of the present invention is preferably a charge transport film (a hole transport layer and an electron transport layer described later), and at least two adjacent layers are more preferably the functional films of the present invention.

Representative element configurations in the organic EL element include, but are not limited to, the following configurations.

(1) Anode/light emitting layer//cathode

(2) Anode/light emitting layer/electron transport layer/cathode
(3) Anode/hole transport layer/light emitting layer/cathode
(4) Anode/hole transport layer/light emitting layer/electron transport layer/cathode
(5) Anode/hole transport laver/light emitting layer/electron transport layer/electron injection layer/cathode
(6) Anode/hole injection layer/hole transport layer/light emitting layer/electron transport layer/cathode
(7) Anode/hole injection layer/hole transport layer/(electron blocking layer/) light emitting layer/(hole blocking layer/) electron transport layer/electron injection layer/cathode

In the above configurations, configuration (7) is preferably used, but not limited thereto.

In the present invention, the hole injection layer, the hole transport layer, etc., may be referred to as “organic functional layer group 1”, and the electron transport layer, electron injection layer, etc., may be referred to as “organic functional layer group 2”.

The light emitting layer used in the present invention is composed of a single layer or a plurality of layers, and in the case of a plurality of light emitting layers, a non-light emitting intermediate layer may be arranged between the light emitting layers.

A hole blocking layer (also referred to as “hole barrier layer”) or an electron injection layer (also referred to as “cathode buffer layer”) may be arranged between the light emitting layer and the cathode, if necessary, and an electron blocking layer (also referred to as an “electron barrier layer”) or a hole injection layer (also referred to as an “anodic buffer layer”) may be arranged between the light emitting layer and the anode.

The electron transport layer used in the present invention is a layer having a function of transporting electrons, and in a broad sense, an electron injection layer and a hole blocking layer are included in the electron transport layer. Moreover, it may be composed of a plurality of layers.

The hole transport layer used in the present invention is a layer having a function of transporting holes, and in a broad sense, a hole injection layer and an electron blocking layer are included in the hole transport layer. Moreover, it may be composed of a plurality of layers.

In the above typical element configurations, the layer excluding the anode and the cathode is also referred to as an “organic layer”.

(Tandem Structure)

Further, the organic EL element may be an element having a so-called tandem structure in which a plurality of light emitting units including at least one light emitting layer are laminated.

As a typical element configuration of the tandem structure, for example, the following configuration can be included.

Anode/1st light emitting unit/intermediate layer/2nd light emitting unit/intermediate layer/3rd light emitting unit/cathode

Here, the first light emitting unit, the second light emitting unit, and the third light emitting unit may all be the same or different. Further, the two light emitting units may be the same, and the remaining one may be different.

The plurality of light emitting units may be directly laminated or may be laminated with an intermediate layer interposed therebetween, and the intermediate layer is generally also called an intermediate electrode, an intermediate conductive layer, a charge generation layer, an electron extraction layer, a connection layer, or an intermediate insulation layer, and publicly known material configurations can be employed provided that they are each a layer having a function of supplying electrons to the adjacent layer on the anode side and holes to the adjacent layer on the cathode side.

Materials used for the intermediate layer include, for example, conductive inorganic compound layers such as ITO (indium tin oxide), IZO (indium zinc oxide), ZnO₂, TiN, ZrN, HfN, TiOₓ, VOₓ, CuI, InN, GaN, CuAlO₂, CuGaO₂, SrCu₂O₂, LaB₆, RuO₂, Al, bilayer films such as Au/Bi₂O₃, multilayer films such as SnO₂/Ag/SnO₂, ZnO/Ag/ZnO, Bi₂O₃/Au/Bi₂O₃, TiO₂/TiN/TiO₂, TiO₂/ZrN/TiO₂, fullerenes such as C₆₀, conductive organic substance layers such as oligothiophenes, conductive organic compound layers such as metal phthalocyanines, metal-free phthalocyanines, metal porphyrins, metal-free porphyrins, etc., however, the present invention is not limited thereto.

Preferred configurations in the light emitting unit include, for example, configurations in which the anode and the cathode are removed from configurations (1) to (7) in the above typical element configurations, however, the present invention is not limited thereto.

Specific examples of the tandem organic EL element include, the element configurations and constituent materials described in, for example, U.S. Pat. Nos. 6,337,492, 7,420,203, 7,473,923, 6,872,472, and 6,107,734, 6,337,492, WO2005/009087, JP 2006-228712A, JP 2006-24791A, JP 2006-49393A, JP 2006-49394A, JP 2006-49396A, JP 2011-96679A, JP 2005-340187A, JP 4711424, JP 3496681, JP 3884564, JP 4213169, JP 2010-192719A, JP 2009-076929A, JP 2008-078414A, IP 2007-059848A, JP 2003-272860A, JP 2003-045676A, WO2005/094130, etc., but the present invention is not limited thereto.

Further, each layer configuring the organic EL element will be described.

[Substrate]

The substrate applicable to the organic EL element is not particularly limited, and includes, for example, types such as glass and plastic.

The substrate used in the present invention may be light-transparent or light-impermeable. The substrate applicable to the present invention is not particularly limited, and includes, for example, a resin substrate, a thin film metal foil, and a thin flexible glass.

The resin substrate applicable to the present invention includes, for example, polyesters such as polyethylene terephthalate (abbreviation: PET) and polyethylene naphilialate (abbreviation: PEN), polyethylene, polypropylene, cellophane, cellulose esters such as cellulose diacetate, cellulose triacetate (abbreviation: TAC), cellulosic acetate butyrate, cellulose acetate propionate (abbreviation: CAP), cellulose acetate phthalate, cellulose nitrate, and derivatives thereof, etc., polyvinylidene chloride, polyvinyl alcohol, polyethylene vinyl alcohol, syndiotactic polystyrene, polycarbonate (abbreviation: PC), norbornene resins, polymethylpentene, polyether ketone, polyimide, polyethersulfone (abbreviation: PES), polyphenylene sulfide, polysulfones, polyetherimide, polyetherketoneimide, polyamide, fluororesins, nylon, polymethylmethacrylate, acrylic and polyarylates, cycloolefin resins such as Arton (trade name, manufactured by JSR Corporation) and Apel (trade name, manufactured by Mitsui Chemicals Inc.).

Among these resin substrates, films such as polyethylene terephthalate (abbreviation: PET), polybutylene terephthalate, polyethylene naphthalate (abbreviation: PEN), and polycarbonate (abbreviation: PC) are preferably used as resin substrates that are flexible in terms of cost and availability.

The thickness of the resin substrate is preferably in the range of 3 to 200 μm as a thin-film resin substrate, more preferably in the range of 10 to 150 μm, and particularly preferably in the range of 20 to 120 μm.

Moreover, the thin plate glass that can be applied as the substrate used in the present invention is a glass plate that is thin enough to be curved. The thickness of the thin plate glass can be appropriately set within a range in which the thin plate glass exhibits flexibility.

The thin plate glass includes, for example, soda lime glass, barium•strontium-containing glass, lead glass, aluminosilicate glass, borosilicate glass, barium borosilicate glass, quartz, etc. The thickness of the thin plate glass is, for example, in the range of 5 to 300 μm, preferably in the range of 20 to 150 μm.

Further, the material for forming the thin-film metal foil includes, for example, one or more types of metals or alloys selected from the group consisting of stainless steel, iron, copper, aluminum, magnesium, nickel, zinc, chromium, titanium, molybdenum, silicon, germanium and tantalum. The thickness of the thin-film metal foil can be appropriately set within a range in which the thin-film metal foil exhibits flexibility, and is, for example, in the range of 10 to 100 μm, preferably in the range of 20 to 60 μm.

(1st Electrode: Anode)

The anode configuring the organic EL element includes metals such as Ag and Au, or an alloy containing a metal as a main component, CuI, or metal oxides such as an indium-tin composite oxide (ITO), SnO₂ and ZnO, and it is preferably a metal or an alloy containing a metal as a main component, and more preferably silver or an alloy containing silver as a main component.

When the transparent anode is composed mainly of silver, the purity of silver is preferably 99% or more. Further, palladium (Pd), copper (Cu), gold (Au), etc., may be added to ensure the stability of silver.

The transparent anode is a layer composed mainly of silver, and specifically, it may be formed of silver alone or may be composed of an alloy containing silver (Ag). Such an alloy includes, for example, silver magnesium (Ag•Mg), silver═copper (Ag•Cu), silver•palladium (Ag•Pd), silver•palladium•copper (Ag•Pd•Cu), and silver•indium (Ag•In), etc.

Among each constituent material constituting the aforementioned anode, the anode constituting the organic EL element used in the present invention is preferably a transparent anode composed mainly of silver and having a thickness in the range of 2 to 20 nm, and more preferably in the range of 4 to 12 nm. When the thickness is 20 nm or less, the absorption component and the reflection component of the transparent anode are inhibited to a low level, and a high light transmittance is maintained, which is preferred.

The layer composed of silver as a main component in the present invention refers to a layer containing the silver content in the transparent anode in an amount of 60% by weight or more, preferably in the silver content of 80% by weight or more, more preferably in the silver content of 90% by weight or more, and particularly preferably in the silver content of 98% by weight or more. Further, “transparent” in the transparent anode according to the present invention refers to a light transmittance at a wavelength of 550 nm being 50% or more.

The transparent anode may have a structure in which a layer composed mainly of silver is divided into a plurality of layers and laminated as needed.

Moreover, in the present invention, when the anode is a transparent anode composed mainly of silver, an underlayer is preferably disposed under the transparent anode from the viewpoint of improving the uniformity of the silver film of the transparent anode to be formed. The underlayer is not particularly limited, but is preferably a layer containing an organic compound having a nitrogen atom or a sulfur atom, and a method for forming a transparent anode on the underlayer is a preferred embodiment.

[Light Emitting Layer]

The light emitting layer constituting the organic EL element, which is a phosphorescence-emitting compound or a fluorescent compound, can be used as the light emitting material, but in the present invention, in particular a structure containing a phosphorescence-emitting compound as the light emitting material is preferably used.

This light emitting layer is a layer in which electrons injected from the electrode or the electron transport layer and holes injected from the hole transport layer, recombine to emit light, and the light emitting portion may be in the layer of the light emitting layer or at the interface between the light emitting layer and the adjacent layer.

The configuration of such a light emitting layer is not particularly limited provided that the contained light emitting materials satisfy the light emitting requirements. Further, there may be a plurality of layers having the same emission spectrum and emission maximum wavelength. In this case, a non-light emitting intermediate layer between each light emitting layer is preferably present.

The total thickness of the light emitting layer is preferably in the range of 1 to 100 nm and more preferably in the range of 1 to 30 nm because a lower drive voltage can be obtained. The total thickness of the light emitting layer is the thickness including the intermediate layer when the non-light emitting intermediate layer is present between the light emitting layers.

The light emitting layer as described above can be formed by using a light emitting material or a host compound described later by, for example, publicly known methods such as a vacuum deposition method, a spin coating method, a casting method, an LB method (Langmuir Blodgeit method), or an inkjet method.

Further, in the light emitting layer, a plurality of light emitting materials may be mixed, and a phosphorescence-emitting material and a fluorescent-emitting material (also referred to as “fluorescent dopant” or “fluorescent compound”) may be fluxed and used in the same light emitting layer. The light emitting layer configuration preferably comprises a host compound (also referred to as “light emitting host”, etc.) and a light emitting material (also referred to as “light emitting dopant compound”) and emits light from the light emitting material.

(Host Compound)

The host compound contained in the light emitting layer is preferably a compound having a phosphorescence quantum yield of phosphorescent emission at room temperature (25° C.) of less than 0.1. Further, the phosphorescence quantum yield is preferably less than 0.01. Moreover, among the compounds contained in the light emitting layer, the volume ratio in the layer is preferably 50% or more.

As the host compound, a publicly known host compound may be used alone, or a plurality of types of host compounds may be used. By using a plurality of types of host compounds, it is possible to adjust the movement of electric charges, and to improve the efficiency of the organic electroluminescence element. Further, by using a plurality of types of light emitting materials described later, it is possible to mix different emitted lights, whereby an arbitrary light emitting color can be obtained.

The host compound used in the light emitting layer may be a conventionally known low molecular compound or a high molecular compound having a repeating in and a low molecular compound having a polymerizable group such as a vinyl group or an epoxy group (vapor-deposited polymerizable light emitting host).

The host compound applicable to the present invention includes compounds described in, for example, JP 2001-257076A, JP 2001-357977A, JP 2002-8860A, JP 2002-43056A, JP 2002-105445A, JP 2002-352957A, JP 2002-231453A, JP 2002-234888A, JP 2002-260861A, JP 2002-305083A, USP 2005/0112407A1, USP 2009/0030202A1, WO2001/039234, WO2008/056746, WO2005/089025, WO2007/063754, WO2005/030900, WO2009/086028, WO2012/023947, JP 2007-254297A, EP 2034538.

(Light Emitting Material)

The light emitting materials that can be used in the present in include a phosphorescence-emitting compound (also referred to as “phosphorescent compound”, “phosphorescence-emitting material or phosphorescence-emitting dopant”) and fluorescence-emitting compound (“fluorescent compound” or “fluorescence-emitting material”), and in particular the phosphorescence-emitting compound is preferably used in order to obtain high luminous efficiency.

<Phosphorescence-Emitting Compound>

The phosphorescence-emitting compound is a compound in which light emission from the excited triplet is observed, specifically, it is defined as a compound that emits phosphorescence at room temperature (25° C.), and the phosphorescence quantum yield is 0.01 or more at 25° C., and it is more preferably 0.1 or more.

The phosphorescence quantum yield can be measured by the method described on page 398 of Spectroscopy II of the 4th edition Experimental Chemistry Course 7 (1992 edition, published by Maruzen Co., Ltd.). The phosphorescence quantum yield in a solution can be measured using various solvents, but when the phosphorescence-emitting compound is used in the present invention, the above phosphorescence quantum yield any of the solvents that is 0.01 or more may be acceptable.

The phosphorescence-emitting compound can be appropriately selected from publicly known compounds used for the light emitting layers of general organic EL elements, and it is preferably a complex-based compound containing a metal of Group 8 to 10 in the periodic table of the element, more preferably an iridium compound, an osmium compound, a platinum compound (platinum complex-based compound) or a rare earth complex, and most preferably the iridium compound.

In the present invention, at least one light emitting layer may contain two or more types of phosphorescence-emitting compounds, and the light emitting layer may have a mode whereby the concentration ratio of the phosphorescence-emitting compounds therein changes in the thickness direction of the light emitting layer.

Specific examples of publicly known phosphorescence-emitting compound that can be used in the present invention include compounds described in the following literatures.

The compounds described in the literatures, such as Nature 395, 151 (1998), Appl. Phys. Lett. 78, 1622 (2001), Adv. Mater. 19, 739 (2007), Chem. Mater, 17, 35:32 (2005), Adv, Mater, 17, 1059 (2005), WO2009/100991, WO2008/101842, WO2003/040257, USP 2006/835469A1, USP 2006/0202194A1, USP 2007/0087321A1, USP 2005/0244673A1, etc., are included.

Further, the compounds described in the literatures, such as Inorg. Chem. 40, 1704 (2001), Chem. Mater. 16, 2480 (2004), Adv. Mater. 16, 2003 (2004), Angew. Chem. Int. Ed. 2006, 45, 7800, Appl. Phys. Lett. 86, 153505 (2005), Chem. Lett. 34, 592 (2005), Chem. Commun. 2906 (2005), Inorg. Chem, 42, 1248 (2003), WO2009/050290, WO2009/000673, U.S. Pat. No. 7,332,232, USP 2009/0039776A1, U.S. Pat. No. 6,687,266, USP 2006/0008670A1, USP 2008/0015355A1, U.S. Pat. No. 7,396,598, USP 2003/013865 7A1, and U.S. Pat. No. 7,090,928, are included.

Moreover, the compounds described in the literatures, such as, Angew. Chem. Int. Ed. 47, 1 (2008), Chem. Mater. 18, 5119 (2006), Inorg. Chem. 16, 4308 (2007), Organometallics 23, 3745 (2004), Appl. Phys. Lett, 74, 1361 (1999), WO2006/056418, WO2005/123873, WO2006/082742, USP 2005/0260441A1, U.S. Pat. No. 7,534,505, USP 2007/0190359A1, U.S. Pat. Nos. 7,338,722, 7,279,704, and USP 2006/103874A1, are also included.

Furthermore, the compounds described in the literatures, such as WO2005/076380, WO2008/140115, WO2011/134013, WO2010/086089, WO2012/020327, WO2011/051404, WO2011/073149, JP 2009-114086A, JP 2003-81988A, and JP 2002-363552A, are also included.

In the present invention, preferred phosphorescence-emitting compounds include organometallic complexes having Ir as the central metal. A complex containing at least one coordination mode of a metal-carbon bond, a metal-nitrogen bond, a metal-oxygen bond, and a metal-sulfur bond is more preferred.

The phosphorescence-emitting compound described above (also referred to as “phosphorescence-emitting metal complex”) can be synthesized by applying the method disclosed in, for example, Organic Letter, Vol. 3, No. 16, pp. 2579-2581 (2001), inorganic Chemistry, Vol. 30, No. 8, pp. 1685-1687 (1991), J. Am. Chem. Soc., Vol. 123, p. 4304 (2001), Inorganic Chemistry, Vol. 40, No. 7, pp. 1704-1711 (2001), inorganic Chemistry, Vol. 41, No. 12, pp. 3055-3066 (2002), New Journal of Chemistry, Vol. 26, p. 1171 (2002), European Journal of Organic Chemistry, Vol. 4, pp. 695-709 (2004), and the references described in these literatures.

<Fluorescence-Emitting Compound>

Fluorescence-emitting compounds include coumarin-based dyes, pyran-based dyes, cyanine-based dyes, croconium-based dyes, squarylium-based dyes, oxobenzanthracene-based dyes, fluorescein-based dyes, rhodamine-based dyes, pyrylium-based dyes, perylene-based dyes, stilbene-based dyes, polythiophene-based dyes, or rare earth complex-based fluorescent material, etc.

Moreover, in recent years, the use of a benzonitrile derivative having a carbazole ring group has been proposed as a host material or a light emitting material for an organic EL element (for example, a thermally activated delayed fluorescence (TADF) compound that emits blue light). Blue light-emitting TADF compounds having a benzonitrile skeleton, such as 2CzPN (dicarbazolyl phthalonitrile: 4,5-di(9H-carbazol-9-yl)phthalonitrile) and 4CzIPN (tetracarbazolisophthalonitrile: 2,4,5,6-tetra(9H-carbazol-9-yl)isophtalonitrile) and 5CzBN (pentacarbazolyl benzonitrile: 2,3,4,5,6-pentakis(carbazol-9-yl)benzonitrile), etc., are known and can be preferably used.

[Organic Functional Layer Groups]

Next, each layer constituting organic functional layer groups 1 and 2 will be described in the order of the charge injection layer, the hole transport layer, the electron transport layer, and the blocking layer.

(Charge Injection Layer)

The charge injection layer is a layer arranged between the electrode and the light emitting layer in order to reduce the drive voltage and improve the light emission brightness. The details are described in “Electrode Materials”, Volume 2, Chapter 2, pages 123 to 166 of “Organic EL Elements and Their industrialization Frontline (published by NTS Inc., Nov. 30,1998)”, and a hole injection layer and an electron injection layer are described therein.

In general the charge injection layer can be present between the anode and the light emitting layer or the hole transport layer in the case of the hole injection layer, and between the cathode and the light emitting layer or the electron transport layer in the case of the electron injection layer; however, the present invention is characterized in that the charge injection layer is arranged adjacent to the transparent electrode. Moreover, when used as an intermediate electrode, at least either of the adjacent electron injection layer and hole injection layer may satisfy the requirements of the present invention.

The hole injection layer is a layer arranged adjacent to the anode that is a transparent electrode, in order to reduce the drive voltage and improve the light emission brightness, the details of which are described in “Electrode Materials”, Volume 2, Chapter 2, pages 123 to 166 of “Organic EL Elements and Their Industrialization Frontline (published by NTS Inc., Nov. 30, 1998)”.

The details of the hole injection layer are also described in JP 9-45479A, JP 9-260062A, JP 8-288069A, etc., and the material used for the hole injection layer includes, for example, porphyrin derivatives, phthalocyanine derivatives, oxazole derivatives, oxadiazole derivatives, triazole derivatives, imidazole derivatives, pyrazoline derivatives, pyrazolone derivatives, phenylenediamine derivatives, hydrazone derivatives, stilbene derivatives, polyarylalkane derivatives, triarylamine derivatives, carbazole derivatives, indrocarbazole derivatives, isoindole derivatives, acene-based derivatives such as anthracene and naphthalene, fluorine derivatives, fluorenone derivatives, and polymer materials or oligomers with polyvinylcarbazole and aromatic amines introduced into the main and side chains, polysilanes, and conductive polymers or oligomers (for example, PEDOT (polyethylene dioxythiophene): PSS (polystyrene sulfonic acid), aniline-based copolymers, polyaniline, polythiophene, etc.).

The triarylamine derivative includes a benzidine type represented by α-NPD (4,4′-bis[N-(1-naphthyl)-N-phenylamino]biphenyl) and a starburst type represented by MTDATA (4,4′,4″-tris[N-(3-methylphenyl)-N-phenylamino]triphenylamine), a compound having fiuorene or anthracene in the triarylamine connecting core portion, etc.

Further, hexaazatriphenylene derivatives as described JP 2003-519432A and JP 2006-135145A can also be used as the hole transport material in the same manner.

The electron injection layer is a layer arranged between the cathode and the light emitting layer in order to reduce the drive voltage and improve the light emission brightness, and when the cathode is composed of the transparent electrode according to the present invention, it is arranged adjacent to the transparent electrode, the details of which are described in “Electrode Materials”, Volume 2, Chapter 2, pages 123 to 166 of “Organic EL Elements and Their Industrialization Frontline (published by NTS Inc., Nov. 30, 1998)”.

The details of the electron injection layer are also described in JP 6-325871A, JP 9-17574A, JP 10-74586A, etc., and specific examples of materials preferably used for the electron injection layer include, metals represented by strontium, aluminum, etc., alkali metal compounds represented by lithium fluoride, sodium fluoride, potassium fluoride, etc., alkali metal halide layers represented by magnesium fluoride, calcium fluoride, etc., an alkaline earth metal compound layer represented by magnesium fluoride, metal oxides represented by molybdenum oxide, aluminum oxide, etc., any a metal complex represented by lithium 8-hydroxyquinolate (Liq), etc. Moreover, when the transparent electrode in the present invention is a cathode, organic materials such as a metal complex are particularly preferably used. The electron injection layer is preferably a very thin film, and the layer thickness is preferably in the range of 1 nm to 10 μm, depending on the constituent materials.

(Hole Transport Layer)

The hole transport layer is composed of a hole transport material having a function of transporting holes, and in a broad sense, the hole injection layer and the electron blocking layer also have the function of the hole transport layer. The hole transport layer can be arranged as a single layer or a plurality of layers.

The hole transport material has either of hole injection or transport and electron barrier property, and may be either an organic substance or an inorganic substance. For example, it includes triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline-based copolymers, conductive polymer oligomers, thiophene oligomers, etc.

As the hole transport material, the compounds described above can be used, but porphyrin compounds, aromatic tertiary amine compounds and styrylamine compounds can be used, and in particular aromatic tertiary amine compounds can be used.

Typical examples of aromatic tertiary amine compounds and styrylamine compounds include N,N,N′,N′-tetraphenyl-4,4′-diaminophenyl, N,N′-diphenyl-N,N′-bis(3-methylphenyl)-[1,1′-biphenyl]-4,4′-diamine (abbreviation: TPD), 2,2-bis(4-di-p-tolylaminophenyl)propane, 1,1-bis(4-di-p-tolylaminophenyl) cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quadriphenyl, N,N,N,-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbenzene, N-phenylcarbazole, etc.

The hole transport layer can be formed by making it into a thin film by publicly known methods such as a vacuum deposition method, a spin coating method, a casting method, a printing method including an inkjet method, and an LB method (Langmuir Blodgett method). The layer thickness of the hole transport layer is not particularly limited, but is usually in the range of about 5 nm to 5 μm and preferably in the range of 5 to 200 nm. The hole transport layer may have a single-layer structure composed of one or more of the above materials.

Moreover, the p property can be enhanced by doping the material of the hole transport layer with impurities. Examples thereof include the materials described in JP 4-297076A, JP 2000-196140A, IP 2001-102175A, and J. Appl. Phys., 95, 5773 (2004), etc.

Such a high p properly of the hole transport layer obtained in this way is preferred because an element having lower power consumption can be fabricated.

(Electron Transport Layer)

The electron transport layer is composed of a material having a function of transporting electrons, and in a broad sense, an electron injection layer and a hole blocking layer are also included in the electron transport layer. The electron transport layer can be arranged as a single-layer structure or a multi-layer laminated structure.

In the electron transport layer having a single layer structure and the electron transport layer haying a laminated structure, an electron transport material (also serving as a hole blocking material) constituting a layer portion adjacent to the light emitting layer may have a function of transmitting electrons injected from the cathode to the light emitting layer. Such a material can be selected and used from any of conventionally known compounds. Examples thereof include nitro-substituted fluorene derivatives, diphenylquinone derivatives, thiopyrandioxide derivatives, carbodiimides, fluorenylidene methane derivatives, anthraquino dimethanes, anthrone derivatives and oxadiazole derivatives. Further, in the above oxadiazole derivatives, a thiadiazole derivative in which the oxygen atom of the oxadiazole ring is substituted with a sulfur atom, and a quinoxaline derivative having a quinoxaline ring known as an electron-withdrawing group, can also be used as materials for the electron transport layer. Further, a polymer material in which these materials are introduced into a polymer chain or a polymer material having these materials in the backbone chain, can also be used.

Moreover, a metal complex of 8-quinolinol derivatives, for example, tris(8-quinolinol)aluminum (abbreviation: Alq₃), tris(5,7-dichloro-8-quinolinol)aluminum, tris(5,7-dibromo-8-quinolinol)aluminum, tris(2-methyl-8-quinolinol)aluminum, tris (5-methyl-8-quinolinol)aluminum, bis(8-quinolinol)zinc (abbreviation: Znq), etc., and a metal complex in which the central metal of these metal complexes is substituted with In, Mg, Cu, Ca, Sn, Ga or Pb, can also be used as a material for the electron transport layer.

The electron transport layer can be formed by making the above materials into a thin film by publicly known methods, such as a vacuum deposition method, a spin coating method, a casting method, a printing method including an inkjet method, an LB method, etc. The layer thickness of the electron transport layer is not particularly limited, but is usually in the range of about 5 nm to 5 μm, preferably in the range of 5 to 200 nm. The electron transport layer may have a single structure composed of one or more of the above materials.

(Blocking Layer)

The blocking layer includes a hole blocking layer and an electron blocking layer, which are layers arranged as necessary in addition to each constituent layer of the organic functional layer unit 3 described above. For example, the blocking layer includes layers described in JP-11-204258A, JP 11-204359A, and a hole blocking (hole block) layer described at page 237 of “Organic EL Elements and Their Industrialization Frontline (published by NTS Inc., Nov. 30, 1998)”, etc.

The hole blocking layer has the function of an electron transport layer in a broad sense. The hole blocking layer is made of a hole blocking material having a significantly small hole transportability while having a function of transporting electrons, and blocking holes while transporting electrons improves the probability of recombination of electrons and holes. In addition, the configuration of the electron transport layer can be used as a hole blocking layer, if necessary. The hole blocking layer is preferably arranged adjacent to the light emitting layer.

On the other hand, the electron blocking layer has a function of a hole transport layer in a broad sense. The electron blocking layer is made of a material having a significantly small electron transportability while having a function of transporting holes, and blocking electrons while transporting holes improves the probability of recombination of electrons and holes. In addition, the configuration of the hole transport layer can be used as an electron blocking layer, if necessary. The layer thickness of the hole blocking layer applied to the present invention is preferably in the range of 3 to 100 nm, and more preferably in the range of 5 to 30 nm.

[2nd Electrode: Cathode]

The cathode is an electrode film that functions to supply holes to the organic functional layer groups and the light emitting layer, and metals, alloys, organic or inorganic conductive compounds or mixtures thereof are used. Specifically, gold, aluminum, silver, magnesium, lithium, magnesium/copper mixture, magnesium/silver mixture, magnesium/aluminum mixture, magnesium/indium mixture, indium, lithium/aluminum mixture, rare earth metal, ITO, ZnO, TiO₂, oxide semiconductors such as SnO₂, etc., are included.

The cathode can be fabricated by forming into a thin film of these conductive materials and their dispersion liquid by methods such as a spin coating method, a casting method, an inkjet method, a vapor deposition method, or a printing method. Moreover, the sheet resistance as the second electrode is preferably several hundred Ω/sq. or less, and the film thickness is usually selected in the range of 5 nm to 5 μm, preferably in the range of 5 to 200 nm.

In the case of the double-sided light emitting type in which the organic EL element also extracts the emitted light L from the cathode side, a cathode having favorable light transmission may be selected and configured.

[Sealing Member]

The sealing means used for sealing the organic EL element includes, for example, a method for bonding the flexible sealing member, the cathode and the transparent substrate with a sealing adhesive.

The sealing member may be arranged so as to cover the display region of the organic EL element, and may be intaglio-shaped or flat-plate-shaped. Moreover, the transparency and electrical insulationability are not particularly limited.

Specific examples include a thin film glass plate, polymer plate, film, and metal film (metal foil), having flexibility. The glass plate includes in particular soda-lime glass, barium•strontium-containing glass, lead glass, aluminosilicate glass, borosilicate glass, barium borosilicate glass, quartz, etc. Further, the polymer plate includes polycarbonate, acrylic, polyethylene terephthalate, polyether sulfide, polysulfone, etc. The metal film includes one or more metals or alloys selected from the group consisting of stainless steel, iron, copper, aluminum, magnesium, nickel, zinc, chromium, titanium, molybdenum, silicon, germanium and tantalum.

In the present invention, a polymer film and a metal film can be preferably used as the sealing member from the viewpoint of being capable of rendering the organic EL element into a thin film. Further, the polymer film preferably has a water vapor transmission rate of 1×10⁻³ g/m²•24 h or less at a temperature of 25±0.5° C. and a relative humidity of 90±2% RH measured by a method compliant with TIS K 7129-1992, and further preferably the oxygen permeability measured by a method compliant with JIS K 7126-1987 of 1×10⁻³ mL/m²•24 h•atom (1 atom is 1.01325×10⁵ Pa) or less, and the water vapor transmission rate at a temperature of 25±0.5° C. and a relative humidity of 90±2% RH of 1×10⁻³ g/m²•24 h or less.

In the gap between the sealing member and the display region (light emitting region) of the organic EL element, an inert gas such as nitrogen or argon or fluorinated hydrocarbon in a gas phase or an inert liquid such as silicone oil in a liquid phase can be injected. Further, the gap between the sealing member and the display region of the organic EL element can be evacuated, or a hygroscopic compound can be sealed in the gap.

Moreover, the sealing film can also be arranged on the transparent substrate in a state where the film completely covers the light emitting functional layer unit in the organic EL element and exposes the terminal portions of anode (3) that is the first electrode and cathode (6) that is the second electrode in the organic EL element.

Such a sealing film is composed of an inorganic material or an organic material, and in particular materials having a function of inhibiting the infiltration of water, oxygen, etc., for example, inorganic materials such as silicon oxide, silicon dioxide, silicon nitride, etc., are used. Further, in order to improve the embrittlement of the sealing film, a film made of an organic material may be used together with the film made of these inorganic materials to form a laminated structure.

The method for forming these sealing films is not particularly limited, and for example, a vacuum vapor deposition method, a sputtering method, a reactive sputtering method, a molecular beam epitaxy method, a cluster ion beam method, an ion plating method, a plasm polymerization method, an atmospheric-pressure plasma polymerization method, a plasma CVD method, a laser CVD method, a thermal CVD method, a coating method, etc., can be employed.

The sealing materials as described above are arranged so as to expose the terminal portions of anode (3) that is the first electrode and cathode (6) that is the second electrode, in the organic EL element, and to cover at least the light emitting functional layer.

[Method for Producing Organic EL Element]

The method for producing the organic EL element is the method for laminating an anode, an organic functional layer group 1, a light emitting layer, an organic functional layer group 2 and a cathode on a transparent base material to form a laminate.

First, a transparent base material is prepared, and a thin film composed of a desired electrode substance, for example, an anode substance is formed on the transparent base material by the methods such as vapor deposition or sputtering so as to have a film thickness of 1 μm or less, preferably in the range of 10 to 200 nm, to form an anode. At the same time, a connection electrode portion for connecting to an external power source is formed at the anode end portion.

Next, on the anode, the hole injection layer and the hole transport layer configuring the organic functional layer group 1, and the light emitting layer, and the electron transport layer configuring the organic functional layer group 2, etc., are laminated in this order.

Each of these layers can be formed by a spin coating method, a casting method, an inkjet method, a vapor deposition method, a printing method, etc., but the vacuum vapor deposition method is particularly preferable because a homogeneous layer can be easily obtained and pinholes are less likely to be formed. Further, different forming methods may be applied to each layer. When the vapor deposition method is adopted for forming each of these layers, the vapor deposition conditions differ depending on the type of compound used, etc., but in general, preferably each condition is appropriately selected, such as the boat heating temperature of 50 to 450° C., the degree of vacuum of 1×10⁻⁶ to 1×10⁻² Pa, the vapor deposition rate of 0.01 to 50 nm/sec, the substrate temperature of −50 to 300° C., and the layer thickness within a range of 0.1 to 5 μm.

After forming organic functional layer group 2 as described above, a cathode is formed on the upper portion by an appropriate forming method such as a spin coating method, a casting method, an inkjet method, a vapor deposition method, or a printing method. In this case, the cathode is patterned in a shape in which the terminal portion is pulled out from above the organic functional layer group to the peripheral edge of the transparent substrate while maintaining insulation state for the anode by the organic functional layer group.

After forming the cathode, the transparent base material, the anode, the organic functional layer groups, the light emitting layer and the cathode are sealed with a sealing material. Namely, with the terminal portions of the anode and the cathode being exposed, a sealing material that covers at least the organic functional layer groups is arranged on the transparent base material.

The organic EL element can be used as electronic devices, for example, a display device, a display, and various light emitting devices. The light emitting device includes, for example, a lighting device (household lighting, interior lighting of vehicle), a backlight for a clock or a liquid crystal, a signboard advertisement, a traffic light, a light source of an optical storage medium, a light source of an electrophotographic copying machine, a light source of an optical communication processor, a light source for a light sensor, etc., but are not limited thereto, and in particular it can be effectively used as a backlight for a liquid crystal display device and a light source for

[One Aspect of Lighting Device]

An aspect of a lighting device fulfilled with the organic EL element of the present invent on will be described.

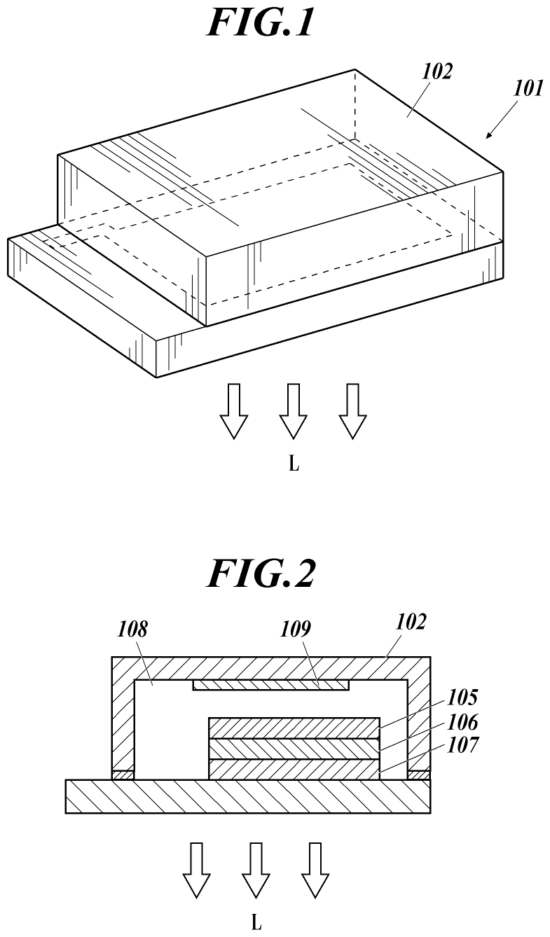

The lighting device as illuminated in FIGS. 1 and 2 can be formed by covering the non-light emitting surface of the organic EL element of the present invention with a glass case, using a glass substrate having a thickness of 300 μm as a sealing substrate and applying an epoxy-based photocurable adhesive (Lux Track LC0629B manufactured by Toa Synthetic Co., Ltd.) as a sealing material around the glass substrate, stacking the glass substrate on the cathode, adhering the substrate to the transparent support substrate, irradiating with UV light from the glass substrate side, to cure the adhesive and seal the inside of the glass case.

FIG. 1 shows a schematic diagram of the lighting device, and the organic EL element (organic EL element 101 in the lighting device) of the present invention is covered with a glass cover 102 (note that the sealing work with the glass cover was carried out in a glove box in a nitrogen atmosphere (of a high-purity nitrogen gas having a purity of 99.999% or more) without the organic EL element 101 in the fighting device being brought into contact with the atmosphere).

FIG. 2 illustrates a cross-sectional view of the lighting device, where 105 is a cathode, 106 is an organic layer, and 107 is a glass substrate with a transparent electrode. Glass cover 102 is filled with nitrogen gas 108, and a water collecting agent 109 is disposed.

By using the organic EL element of the present invention, a lighting device with improved luminous efficiency can be obtained.

### EXAMPLE

Hereinafter, the present invention will be specifically described with reference to Examples, but the present invention is not limited thereto. In the examples, “parts” or “%” indicates “parts by weight” or “% by weight” unless otherwise specified.

First, the compounds used in the Examples were synthesized by the following procedure.

<Synthesis of Exemplified Compound 7>

It was synthesized according to the following scheme.

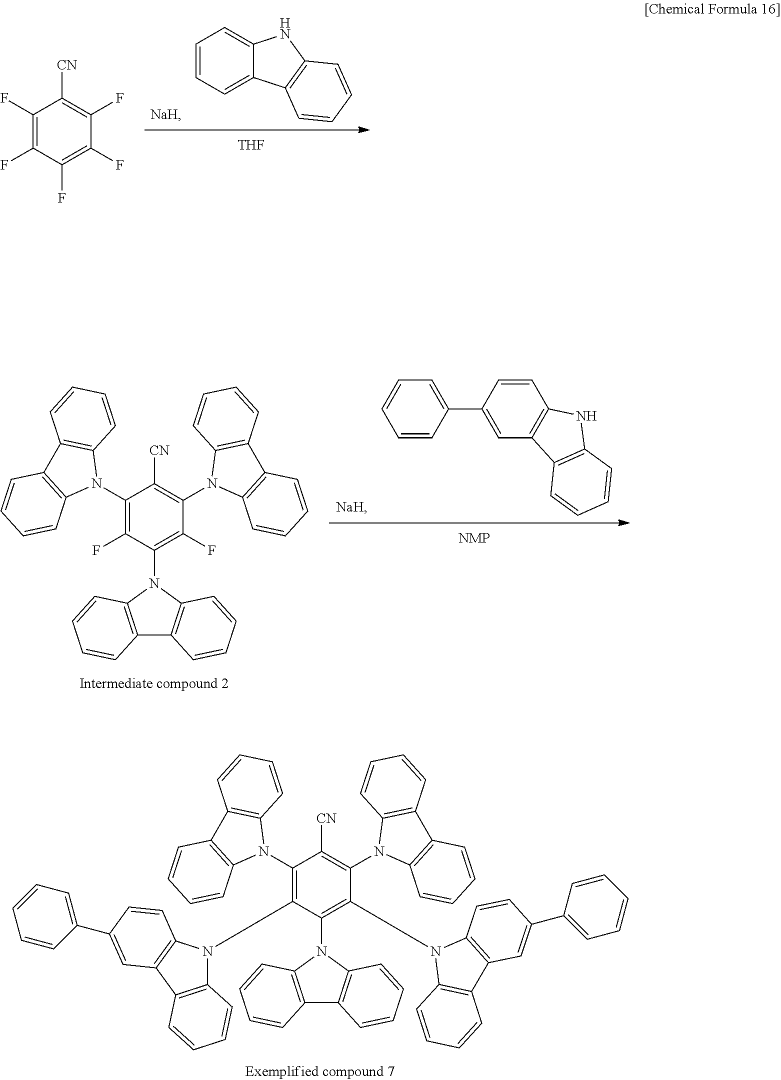

Carbazole (6.47 g, 38.68 mol) was dissolved in THE (tetrahydrofuran) (42 mL), NaH (1.68 g, 42.0 mol) was added, and the mixture was stirred for 30 minutes. Then, 2,3,4,5,6-pentafluorobenzonitrile (1.32 g, 10.8 mol) was added to the solution, and the mixture was stirred with heating under reflux for 5 hours. Following the reaction, water was added to the reaction solution, and the precipitate was collected by filtration. This was recrystallized to obtain 6.51 g of an intermediate.

Next, 3-phenyl-9H-carbazole (5.96 g, 24.5 mot) was dissolved in NMP: N-methylpyrrolidine (42 mL), NaH (0.98 g, 24.5 mol) was added, and the mixture was stirred for 30 minutes. Then, the intermediate (6.51 g, 10.2 mol) was added to the solution, and the mixture was heated and stirred at 120° C. for 5 hours. Water was added to the reaction solution, and the precipitate was collected by filtration. This was recrystallized to obtain 9.93 g of a target exemplified compound (7).

<Synthesis of Exemplified Compound 10>

It was synthesized by the following scheme in the same manner as in above except that the raw material carbazole was mainly changed.

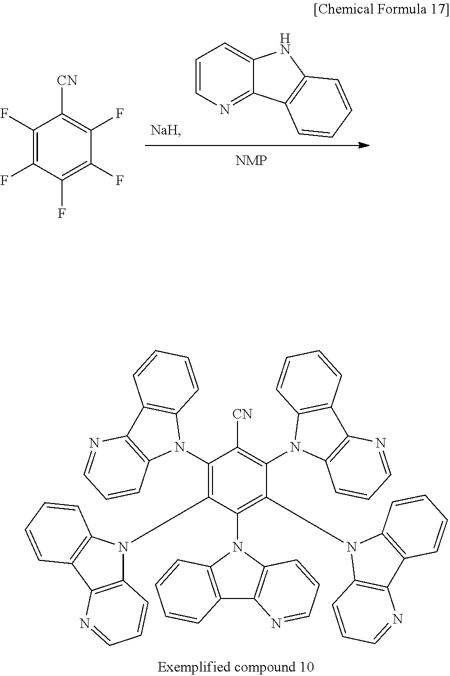

Carboline (10.9 g, 64.6 mol) was dissolved in NMP (42 mL), NaH (2.80 g, 70.0 mol) was added, and the mixture was stirred for 30 minutes. Then, 2,3,4,5,6-pentafluorobenzonitrile (1.32 g, 10.8 mol) was added to the solution, and the mixture was heated and stirred at 120° C. for 5 hours. Water was added to the reaction solution, and the precipitate was collected by filtration. This was recrystallized to obtain 9.20 g of a target exemplified compound (10).

<Synthesis of Exemplified Compound 15>

Exemplified compound 15 was synthesized by the following scheme in the same manner as in above except that the raw material carbazole was mainly changed.

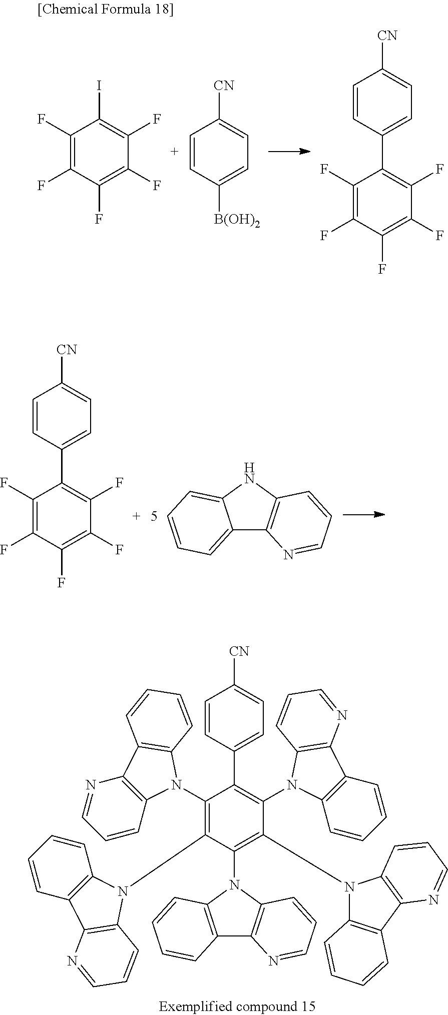

4-cyanophenylboronic acid (1.2 g, 4.1 mmol) and pentafluoroiodobenzene (0.6 g, 4.1 mmol) were dissolved in dioxane (50 mL), Pd₂(dba)₃ (0.2 g, 0.2 mmol), K₂CO₃ (1.7 g, 12.2 mmol) and S-Phos (0.3 g, 0.8 mmol) were added, and the mixture was stirred at 110° C. for 6 hours. Water was added to the reaction solution, and the precipitate was collected by filtration. This was recrystallized to obtain 1.07 g of an intermediate.

Next, 5H-pyrido[3,2-b]indole (4.0 g, 23.9 mmol) was dissolved in THF (42 mL), NaH (1.0 g, 23.9 mmol) was added, and the mixture was stirred for 30 minutes. Then, the intermediate (1.07 g, 4.0 mmol) was added to the solution, and the mixture was heated and stirred at 70° C. for 5 hours. Water was added to the reaction solution, and the precipitate was collected by filtration. This was purified by column chromatography to obtain 3.6 g of a target exemplified compound (15).

### <Synthesis of Other Exemplified Compounds>

Exemplified compounds 1 to 6, 8, 9, and 11 to 14 were synthesized in the same manner as in the synthesis of the above exemplified compounds.

### Example 1

For the following mCP (Sample A) and the following exemplified compound 10 (Sample B), respectively, the density value of the functional film (initial film density) was calculated at 300 K by the molecular dynamics calculation of NPT ensemble, and the film density value of the functional film (film density after storage) was calculated by carrying out the molecular dynamics calculation at 370 K, and they are shown in Table I below.

In addition, the ratio of change in film density was determined as shown below, and is shown in Table I below.

Ratio of change in film density (%)={|initial film density−film density after storage|/initial film density}×100

### (1) Measurement Method of Film Density

The film density specified in the present invention is calculated and obtained by the following method.

<Calculation Software>

Materials Science Suite (manufactured by Schrodinger K.K.) was used.

### <Calculation Procedure>

(a) Create a molecular structure and optimize the structure.
(b) Using the structure optimized in (a), an amorphous structure with an initial film density of 0.5 g/cm³ is created.
(c) Equilibrate the amorphons structure created in (b) by using the following molecular dynamics (MD) calculation conditions.
(d) Obtain the film density (g/cm³) in the obtained cell so as to match the following conditions. The term “cell” as used in the present invention refers to a unit containing the specified number of molecules.

<MD Calculation Conditions>

The calculation time was 10 nanoseconds, the ensemble method was NPT, the pressure was 1 atm, and the number of molecules was 300 molecules. The temperature was 300 K or 370 K.

<Calculation Conditions for Film Density>

For the film density specified in the present invention, when the calculation of 10 nanoseconds was completed, the average value of the final 20% of the trajectory data was taken as the film density. At this time, the standard deviation of the density changing with time was confirmed to be within 5%, which was deemed to be sufficiently equilibrated.

[Table 1]

TABLE I

| | | | Film | Ratio of | |
|---|---|---|---|---|---|
| | | Initial | density | change in | |
| | | film | after | film | |
| Sample | | density | storage | density | |
| No. | Compound | [g/cm3] | [g/cm3] | [%] | Remarks |
| A | mCP | 1.1335 | 1.1198 | 1.2 | Comparative |
| | | | | | Example |
| B | Exemplified | 1.0974 | 1.0951 | 0.2 | Present |
| | Compound 10 | | | | invention |

As is clear from Table 1, it was found that when the compound according to the present invention was used, the initial film density was small and the ratio of change film density after storage was significantly small.

### Example 2

An organic EL element was fabricated by using the above synthesized exemplified compound.

It is noted that the compounds used for producing the organic EL element are as follows.

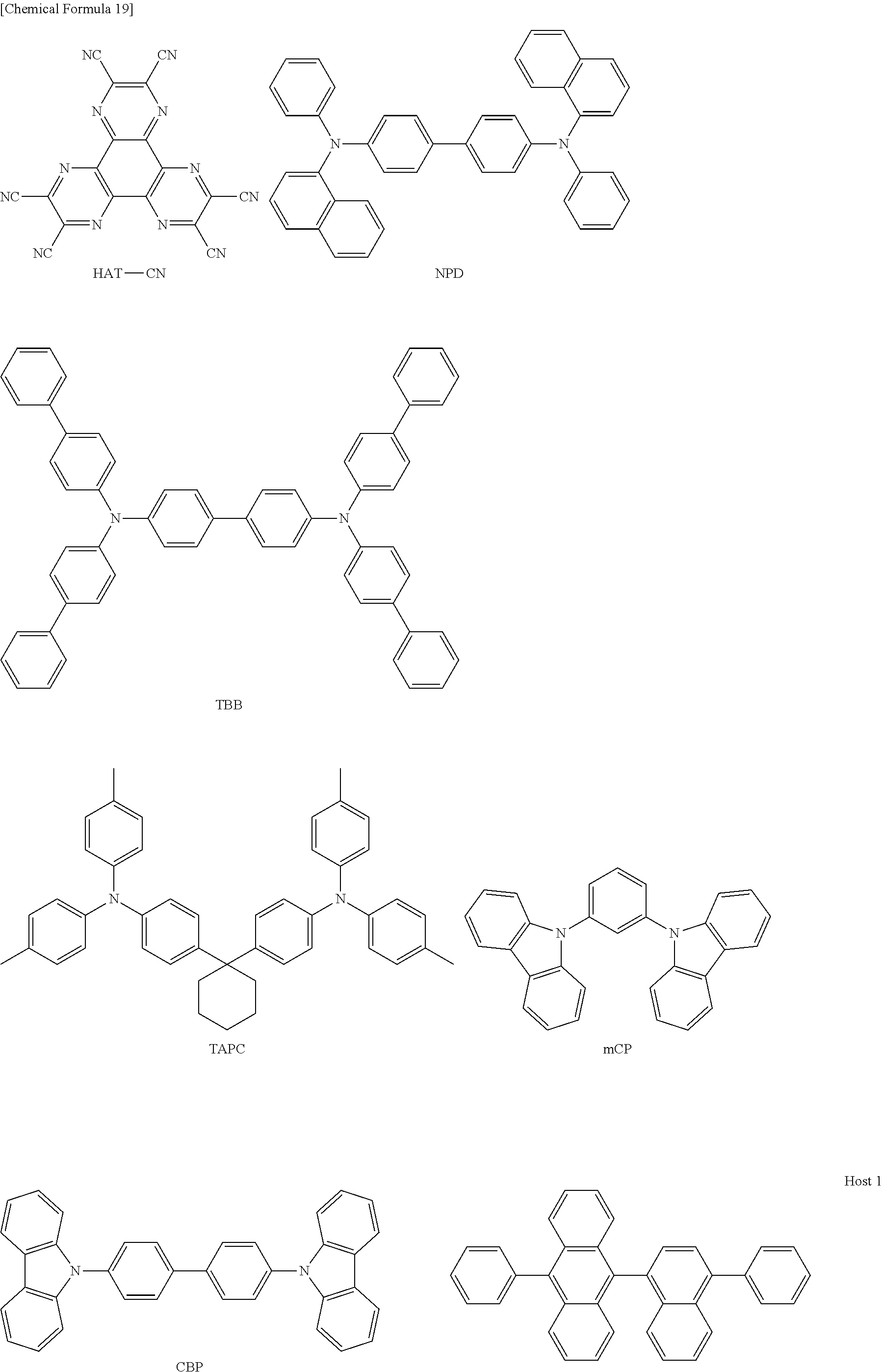

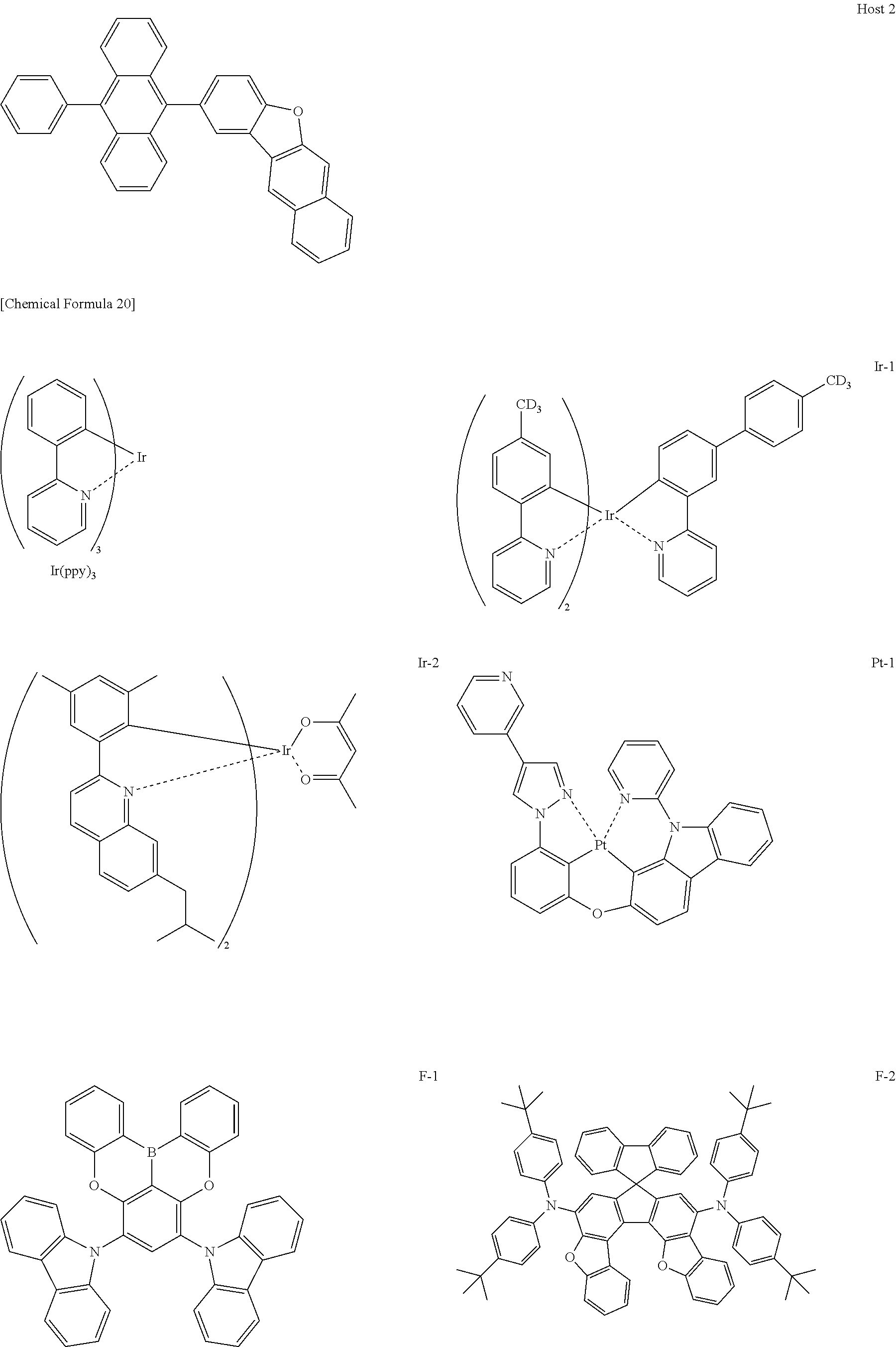

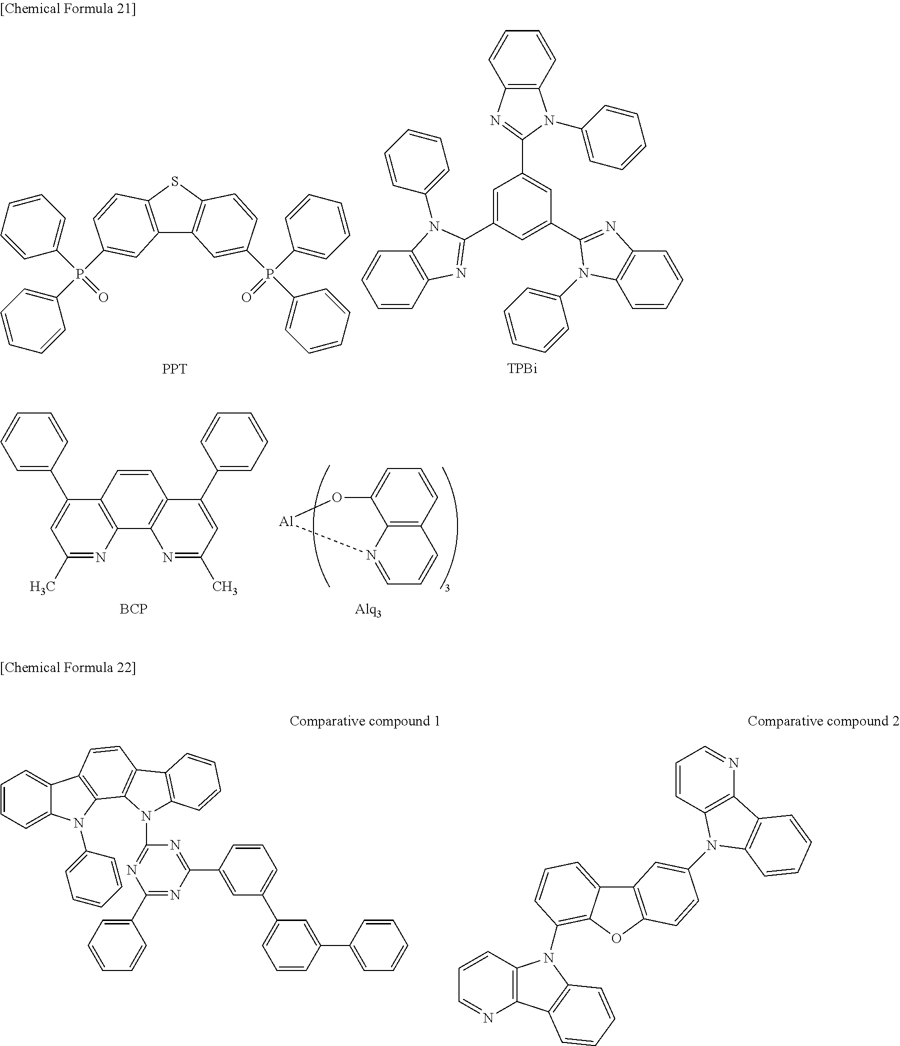

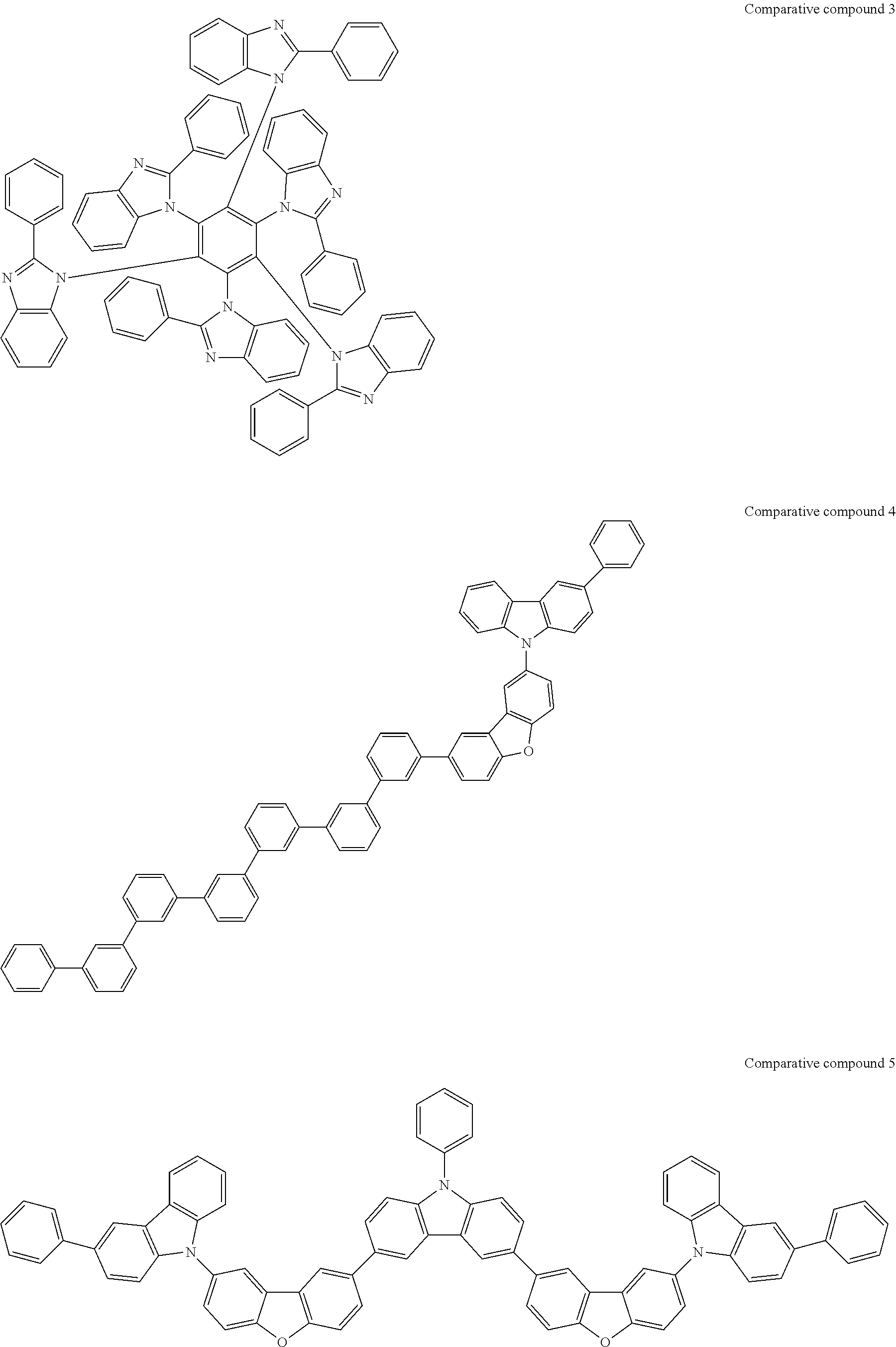 [SMILES: c1ccc(-c2nc3ccccc3n2-c2c(-n3c(-c4ccccc4)nc4ccccc43)c(-n3c(-c4ccccc4)nc4ccccc43)c(-n3c(-c4ccccc4)nc4ccccc43)c(-n3c(-c4ccccc4)nc4ccccc43)c2-n2c(-c3ccccc3)nc3ccccc32)cc1 ; c1ccc(-c2cccc(-c3cccc(-c4cccc(-c5cccc(-c6cccc(-c7cccc(-c8ccc9oc%10ccc(-n%11c%12ccccc%12c%12cc(-c%13ccccc%13)ccc%12%11)cc%10c9c8)c7)c6)c5)c4)c3)c2)cc1 ; c1ccc(-c2ccc3c(c2)c2ccccc2n3-c2ccc3oc4ccc(-c5ccc6c(c5)c5cc(-c7ccc8oc9ccc(-n%10c%11ccccc%11c%11cc(-c%12ccccc%12)ccc%11%10)cc9c8c7)ccc5n6-c5ccccc5)cc4c3c2)cc1]

<Fabrication of Organic EL Element 1-1>

Patterning was carried out on a substrate (NA45 manufactured by AvanStrate Inc.) in which ITO (indium tin oxide) was formed with a film thickness of 100 nm on a glass substrate having a thickness of 100 mm×100 mm×1.1 mm as an anode. Then, the transparent support substrate arranged with the ITO transparent electrode was ultrasonically cleaned with isopropyl alcohol, dried with a dry nitrogen gas, and washed with UV ozone for 5 minutes.

Subsequently, this transparent support substrate was coated with a solution in which poly(3,4-ethylenedioxythiophene)-polystyrene sulfonate (PEDOT/PSS, Baytron P Al 4083, manufactured by Bayer AG) was diluted to 70% with pure water, by a spin coating method under the condition of 3,000 rpm and 30 seconds to form a thin film, and it was dried at 200° C. for 1 hour to arrange a hole injection layer having a layer thickness of 20 nm.

Then, a thin film was formed by a spin coating method under the conditions of 2,000 rpm and 30 seconds by using a solution of polyvinyl carbazole (Mw of about 1,100,000) dissolved in 1,2 dichlorobenzene, and then dried at 120° C. for 10 minutes to arrange a hole transport layer having a layer thickness of 15 nm.

Further, a thin film was formed by a spin coating method, under the conditions of 2,000 rpm and 30 seconds, by using solutions in which Ir (ppy)₃ as a light emitting dopant and comparative compound 1 shown in Table II as a host compound were dissolved in toluene so as to be 10% and 90% by weight, respectively, and then dried at 100° C. for 10 minutes to arrange a light emitting layer having a layer thickness of 35 nm.

Next, this substrate was fixed to the substrate holder of a commercially available vacuum vapor deposition apparatus.

Each of the crucibles for vapor deposition in the vacuum vapor deposition apparatus was filled with the constituent materials of each layer in the optimum amounts for fabricating the element. The crucible for vapor deposition made of molybdenum or tungsten that is used for a resistance heating material was used.

After decompressing to a vacuum degree of 1×10⁻⁴ Pa, TPBi (1,3,5-tris(N-phenylbenzimidazol-2-yl)) was vapor-deposited at the vapor deposition rate of 1.0 nm/sec to form an electron transport layer with a layer thickness of 30 nm.

Further, after forming lithium fluoride with a film thickness of 0.5 nm. aluminum having a thickness of 100 nm was vapor-deposited to form a cathode.

The non-light emitting surface side of the above element was covered with a can-shaped glass case in an atmosphere of a high-purity nitrogen gas having a purity of 99.999% or more, and an electrode extraction wiring was arranged to fabricate an organic EL element 1-1.

<Fabrication of Organic EL Elements 1-2 and 1-3>

Organic EL elements 1-2 and 1-3 were each fabricated in the same manner as the organic EL element 1-1 except that the host compound was changed as shown in Table I below.

«Evaluation of Organic EL Elements 1-1 to 1-3»

When evaluating the obtained organic EL elements 1-1 to 1-3, the lighting device as illustrated in FIGS. 1 and 2 can be formed by covering the non-light emitting surface of the each organic EL element with a can-shaped glass case following fabrication of the cathode, using a glass substrate having a thickness of 300 μm as a sealing substrate and applying an epoxy-based photocurable adhesive (Lux Track LC0629B manufactured by Toa Synthetic Co., Ltd.) as a sealing material around the glass substrate, stacking the glass substrate on the cathode, adhering the substrate to the transparent support substrate, irradiating with UV light from the glass substrate side, to cure the adhesive and seal the inside of the glass case.

The following evaluation was carried out for each sample prepared in this way. The evaluation results are shown in Table II.

(2) Ratio of Change in Film Density After Storage

The film density value of the functional film calculated by carrying out the molecular dynamics calculation under the condition of 370 K was used as the film density value of the functional film stored under the temperature, and the difference from the initial film density is obtained.

Good: The ratio of change in the difference between the initial film density of each element and the film. density value after storage is 1% or less with respect to the initial film density.

Failure: The ratio of change in the difference between the initial film density of each element and the film density value after storage is greater than 1% with respect to the initial film density.

### (3) Drive Voltage

Each of the voltage when organic EL element was driven at room temperature (about 23 to 25° C.) under the constant current condition of 2.5 mA/cm², was measured, and from the measurement result, the relative value with respect to the value of organic EL element 1-1 as 100, was determined as shown below.

Drive voltage=(drive voltage of each element/drive voltage of organic EL element 1-1)×100

It should be noted that the smaller the value, the lower the drive voltage compared to that of the comparative example.

(4) External Extraction Quantum Efficiency (Also Called “Luminous Efficiency”)

By using the above organic EL element, lighting was carried out under the constant current condition of 2.5 mA/cm² at room temperature (about 23 to 25° C.), and by measuring the light emission brightness (L) [cd/m²] immediately after the start of lighting, the external extraction quantum efficiency (η) was calculated.

Here, the light emission brightness was measured by using CS-1000 (manufactured by Konica Minolta Sensing Co., Ltd.), and the external extraction quantum efficiency was defined as the relative value with respect to that of organic EL element 1-1 as 100.

(5) Half-Life

The half-life was evaluated according to the measurement method described below.

Each organic EL element was driven with a constant current at the current imparting an initial brightness of 1,000 cd/m², and the time to become ½ (500 cd/m²) of the initial brightness was obtained, which was defined as a measure of half-life. The half-life was expressed as the relative value with respect to that of organic EL element 1-1 as 100.

(6) Voltage Rise Upon Drive

Each of the voltage when the organic EL element was driven at room temperature (about 23 to 25° C.) under the constant current condition of 2.5 mA/cm² was measured, and the voltage rise upon drive was calculated from the measurement results by the following formula. The voltage rise upon drive was represented by the relative value with respect to that of organic EL element 1-1 as 100.

Voltage rise upon drive=Drive voltage at half brightness of the initial brightness−initial drive voltage

It should be noted that the smaller the value, the smaller the voltage rise upon drive compared to that of the Comparative Example.

TABLE 2
Table II

| Organic | | | Ratio of | | | | | |
|---|---|---|---|---|---|---|---|---|
| EL | | Initial film | change in | | | | Change | |
| element | | density | density after | Drive | Luminous | Element | in drive | |
| No. | Compound | [g/cm³] | storage | voltage | efficiency | life | voltage | Remarks |
| 1-1 | Comparative | 1.1463 | Unacceptable | 100 | 100 | 100 | 100 | Comparative |
| | Compound 1 | | | | | | | Example |
| 1-2 | Exemplified | 1.1577 | Good | 95 | 110 | 130 | 91 | Present |
| | Compound 1 | | | | | | | invention |
| 1-3 | Exemplified | 1.0817 | Good | 90 | 119 | 156 | 78 | Present |
| | Compound 2 | | | | | | | invention |

As is clear from Table II, it was found that when using the compound according to the present invention, the initial film density was small and the ratio of change in film density after storage was significantly small.

Furthermore, it was found that the luminous efficiency and light emission life were superior to those of the organic EL element of the Comparative Examples, the drive voltage was clearly low, and the voltage rise upon drive was inhibited.

### Example 3

### <Fabrication of Organic EL Element 2-1>

Patterning was carried out on a substrate (NA-45 manufactured by AvanStrate Inc.) in which ITO (indium tin oxide) was formed as an anode on a glass substrate of 100 mm×100 mm×1.1 mm to a thickness of 100 nm. Then, the transparent support substrate arranged with the ITO transparent electrode was ultrasonically cleaned with isopropyl alcohol, dried with a dry nitrogen gas, and washed with UV ozone for 5 minutes.

This transparent support substrate was fixed to the substrate holder of a commercially available vacuum vapor deposition apparatus, 200 mg of NPD was charged in a molybdenum resistance heating boat as a hole transport material, and 200 mg of F-1 was charged in other molybdenum resistance heating boat as a dopant, 200 mg of comparative compound 2 was charged in other molybdenum resistance heating boat as host compound 1, 200 mg of CBP was charged in other molybdenum resistance heating boat as host compound 2, 200 mg of BCP was charged in other molybdenum resistance heating boat as a hole blocking material, and further 200 mg of Alq₃ was charged in other molybdenum resistance heating boat as an electron transport material, and these boats were attached to the vacuum vapor deposition apparatus.

Next, after decompressing the vacuum chamber to 4×10⁻⁴ Pa, the heating boat containing the NPD was energized and heated, and the vapor deposition was carried out at the vapor deposition rate of 0.1 nm/sec to form a hole transport layer having a layer thickness of 10 nm on the transparent support substrate.

Further, the heating boats containing F-1, CBP and comparative compound 2 were energized and heated, and they were vapor co-deposited on the hole transport layer at the vapor deposition rates of 0.06 nm/sec, 0.20 nm/sec, and 0.74 nm/sec, respectively, to form a light emitting layer having a layer thickness of 40 nm.

Further, the heating boat containing BCP was energized and heated, and vapor deposition was carried out at the vapor deposition rate of 0.1 nm/sec to form a hole blocking layer having a layer thickness of 10 nm on the light emitting layer.

Further, the heating boat containing Alq₃ was energized and heated, and vapor deposition was carried out at the vapor deposition rate of 0.1 nm/sec to form an electron transport layer having a layer thickness of 30 nm on the hole blocking layer.

Further, magnesium and silver were vapor co-deposited on the electron injection layer at a ratio of 10:1 (molar ratio) with a thickness of 100 nm to form a cathode, and to fabricate an organic EL element 2-1.

<Fabrication of Organic EL Elements 2-2 to 2-4>

Organic EL elements 2-2 to 2-4 were each fabricated in the same manner as in the fabrication of organic EL element 2-1 except that comparative compound 2 was changed to the compound shown in Table III.

«Evaluation of Organic EL Elements 2-1 to 2-4»

When evaluating the obtained organic EL elements, organic EL elements 2-1 to 2-4 each was sealed in the same manner as in tile organic EL elements 1-1 to 1-3 of Example 1 and lighting devices as shown in FIGS. 1 and 2 were each fabricated and evaluated.

Each sample fabricated in this manner was subjected to evaluation, as in Example 1, of the initial film density, the ratio of change in film density after storage, the external extraction quantum efficiency, the half-life, the drive voltage, and the voltage rise upon drive. The measurement results of the ratio of change in film density after storage, the external extraction quantum efficiency, the half-life, the drive voltage, and the voltage rise upon drive in Table III were shown as relative values with respect to the measured values of organic EL element 2-1 as 100.

Furthermore, the following evaluation was further carried out for each sample. The above evaluation results are shown in Table III.

(7) Vapor Deposition Reproducibility

Using the same material, an organic EL element was fabricated 10 times by the same method, the half-life of each organic EL element was measured, and then the average value was calculated.

Good: The difference between the average value of the half-life values of the elements and the half-life value of each of the 10 elements is within 10% for all the elements.

Fair: The difference between the average value of the half-life values of the elements and the half-life value of each of the 10 elements is within 20% for all the elements.
Unacceptable: The difference between the average value of the half-life values of the elements and the half-life value of each of the 10 elements is greater than 20% for one or more elements.

### (8) Presence or Absence of Burning on the Vapor Deposition Boat

The molybdenum resistance heating boat containing the aromatic compound according to the present invention, which was used in the fabrication of each organic EL element, was opened after the element fabrication was completed, and the presence or absence of the burning (also referred to as “kogetion”) inside the vapor deposition boat was visually confirmed.

Good: No burning is observed in the molybdenum resistance heating boat after heating.

Fair: Burning is observed in a portion of the molybdenum resistance heating boat after heating.

Unacceptable: Burning is observed in the whole molybdenum resistance heating boat after heating.

[Table 3]

TABLE III

| Organic | | | Ratio of | | | | | | | |
|---|---|---|---|---|---|---|---|---|---|---|
| EL | | Initial film | change in | | | | Change | | Presence or | |
| element | | density | film density | Drive | Luminous | Element | in drive | | absence of | |
| No. | Compound | [g/cm³] | after storage | voltage | efficiency | life | voltage | Reproducibility | burning | Remarks |
| 2-1 | Comparative | 1.1720 | Unacceptable | 100 | 100 | 100 | 100 | Unacceptable | Fair | Comparative |
| | Compound 2 | | | | | | | | | Example |
| 2-2 | Exemplified | 1.2304 | Good | 96 | 105 | 156 | 83 | Fair | Fair | Present |
| | Compound 3 | | | | | | | | | invention |
| 2-3 | Exemplified | 1.0372 | Good | 91 | 109 | 203 | 75 | Good | Good | Present |
| | Compound 4 | | | | | | | | | invention |
| 2-4 | Exemplified | 1.0808 | Good | 85 | 125 | 195 | 79 | Good | Good | Present |
| | Compound 5 | | | | | | | | | invention |

As is clear from Table III, when using the compound according to the present invention for the element fabrication by vapor deposition, decomposition upon heating did not occur, and the element could be fabricated with satisfactory reproducibility. Moreover, it was found that the initial film density was small and the ratio of change in film density after storage was remarkably small.

Furthermore, it was clear that the luminous efficiency and light emission life were superior to those of the organic EL element of the Comparative Examples and the drive voltage was low, and the voltage rise upon drive was found to be inhibited.

The burning of the resistance heating boat was found to be small as well.

### Example 4

### <Fabrication of Organic EL Element 3-1>

Patterning was carried out on a substrate (NA45 manufactured by AvanStrate Inc.) in which ITO (indium tin oxide) was deposited with a thickness of 100 nm on a glass substrate having a size of 100 nm×100 mm×1.1 mm as an anode. Then, the transparent support substrate arranged with the ITO transparent electrode was ultrasonically cleaned with isopropyl alcohol, dried with a dry nitrogen gas, and washed with UV ozone for 5 minutes.

This transparent support substrate was coated by a spin coating method with a solution of poly (3,4-ethylenedioxythiophene)-polystyrene sulfonate (PEDOT/PSS, Baytron P Al 4083, manufactured by Bayer AG,) diluted to 70% with pure water under the conditions of 3,000 rpm and 30 seconds, to form a thin film, and then it was dried at 200° C. for 1 hour to arrange a hole injection layer having a layer thickness of 20 nm.

This transparent support substrate was fixed to the substrate holder of a commercially available vacuum vapor deposition apparatus, and after rough exhaust of this apparatus was carried out by an oil rotary pump, the exhaust was continued until the degree of vacuum in the apparatus was 1.0×10⁻⁴ Pa or less by using a cryopump.

Next, the heating boat containing NPD was energized and heated, and vapor deposition was carried out at the vapor deposition rate of 0.1 nm/sec to form a hole transport layer having a layer thickness of 30 nm on the transparent support substrate.

Further, the heating boats containing Pt-1 and the host 1 were energized and heated, and they were vapor co-deposited on the hole transport layer at the vapor deposition rates of 0.05 nm/sec and 0.95 nm/sec, respectively to form a light emitting layer having a layer thickness of 40 nm.

Further, the heating boat containing comparative compound 2 was energized and heated, and vapor deposition was carried out at the vapor deposition rate of 0.1 nm/sec to form a hole blocking layer having a layer thickness of 10 nm on the light emitting layer.

Further, the heating boat containing Alq₃ was energized and heated, and vapor deposition was carried out at the vapor deposition rate of 0.1 nm/sec to form an electron transport layer having a layer thickness of 30 nm on the hole blocking layer.

Further, after forming lithium fluoride with a film thickness of 0.5 nm, aluminum having a thickness of 100 nm was vapor-deposited to form a cathode.

The non-light emitting surface side of the above element was covered with a can-shaped glass case in an atmosphere of a high-purity nitrogen gas having a purity of 99.999% or more, and an electrode extraction wiring was arranged to fabricate an organic EL element 3-1.

<Fabrication of Organic EL Elements 3-2 to 3-5>

Organic EL elements 3-2 to 3-5 were each fabricated in the same manner as in the fabrication of organic EL element 3-1 except that comparative compound 2 was changed to the compound shown in Table IV.

«Evaluation of Organic EL Elements 3-2 to 3-5»

When evaluating the obtained organic EL elements, EL elements 3-2 to 3-5 were sealed in the same manner as organic EL elements 1-1 to 1-3 of Example 1, and lighting devices as shown in FIGS. 1 and 2 were each fabricated and evaluated.

Each sample fabricated in this manner was subjected to evaluation, as in Example 2, of the initial film density, the ratio of change in density after storage, the external extraction quantum efficiency, the half-life, the drive voltage and the voltage rise upon drive, the vapor deposition reproducibility, the presence or absence of burning of the boat. The measurement results of the ratio of change in density after storage, the external extraction quantum efficiency, the half-life, the drive voltage, and the voltage rise upon drive in Table IV were shown as relative values with respect to the measured values of organic EL element 3-1 as 100.

TABLE 4
Table IV

| Organic | | | | Ratio of | | |
|---|---|---|---|---|---|---|
| EL | | Presence or | Initial film | change in | | |
| element | | absence of | density | film density | Drive | Luminous |
| No. | Compound | chirality | [g/cm³] | after storage | voltage | efficiency |
| 3-1 | Comparative | Absence | 1.1720 | Unacceptable | 100 | 100 |
| | Compound 2 | | | | | |
| 3-2 | Comparative | Presence | 1.0944 | Unacceptable | 99 | 100 |
| | Compound 3 | | | | | |
| 3-3 | Exemplified | Presence | 1.0487 | Good | 98 | 105 |
| | Compound 6 | | | | | |
| 3-4 | Exemplified | Presence | 1.0510 | Good | 90 | 111 |
| | Compound 7 | | | | | |
| 3-5 | Exemplified | Presence | 1.1171 | Good | 87 | 121 |
| | Compound 8 | | | | | |

| Organic | | | | | |
|---|---|---|---|---|---|
| EL | | Change | | Presence or | |
| element | Element | in drive | | absence of | |
| No. | life | voltage | Reproducibility | burning | Remarks |
| 3-1 | 100 | 100 | Unacceptable | Fair | Comparative |
| | | | | | Example |
| 3-2 | 110 | 97 | Fair | Unacceptable | Comparative |
| | | | | | Example |
| 3-3 | 165 | 89 | Good | Good | Present |
| | | | | | invention |
| 3-4 | 199 | 80 | Good | Good | Present |
| | | | | | invention |
| 3-5 | 256 | 75 | Good | Good | Present |
| | | | | | invention |

As is clear from Table IV, when using the compound according to the present invention for the element fabrication by vapor deposition, decomposition upon heating did not occur, and the element could be fabricated with satisfactory reproducibility. Moreover, it was found that the initial film density was small and the ratio of change in film density after storage was remarkably small.

Furthermore, it was clear that the luminous efficiency and light emission life were superior to those of the organic EL elements of the Comparative Examples, the drive voltage was low, and the voltage rise upon drive was found to be inhibited. This is because having chirality does not change the physical characteristics of the molecule itself, and even if the element has multiple optical isomers, changes in film quality can be inhibited without compromising initial performance such as voltage and brightness, enhancing the stability with an elapsed of time.

Similarly, the burning of the resistance heating boat was found to be small.

### Example 5

### <Fabrication of Organic EL Element 4-1>

Patterning was carried out on a substrate (NA45 manufactured by AvanStrate Inc.) in which ITO (indium tin oxide) with a thickness of 100 nm was vapor-deposited on a glass substrate having a size of 100 mm×100 mm×1.1 mm as an anode. Then, the transparent support substrate arranged with the ITO transparent electrode was ultrasonically cleaned with isopropyl alcohol, dried with a dry nitrogen gas, and washed with UV ozone for 5 minutes.

This transparent support substrate was fixed to the substrate holder of a commercially available vacuum vapor deposition apparatus, the vacuum chamber was decompressed to 4×10⁻⁴ Pa, and then the heating boat containing NPD was energized and heated, and the vapor deposition was carried out at the vapor deposition rate of 0.1 nm/sec to form a hole transport layer having a layer thickness of 35 nm on a transparent support substrate.

Further, the heating boat containing mCP was energized and heated, and vapor deposition was carried out at the vapor deposition rate of 0.1 nm/sec to form an electron blocking layer having a layer thickness of 10 nm on the hole transport layer.

Further, the heating boats containing exemplified compound 9 (5CzEN) and mCP were energized and heated, and they were vapor co-deposited on the electron blocking layer at the vapor deposition rates of 0.08 nm/sec and 0.92 nm/sec, respectively, to form a light emitting layer having a layer thickness of 15 nm.

Further, the heating boat containing PPT was energized and heated, and vapor deposition was carried out at the vapor deposition rate of 0.1 nm/sec to form a hole blocking layer having a layer thickness of 10 nm on the light emitting layer.

Further, the heating boat containing comparative compound 2 was energized and heated, and vapor deposition was carried out at the vapor deposition rate of 0.1 nm/sec to form an electron transport layer having a layer thickness of 40 nm on the hole blocking layer.

Further, after forming lithium fluoride with a film thickness of 0.8 nm, aluminum having a thickness of 100 nm was vapor-deposited to form a cathode.

The non-light emitting surface side of the above element was covered with a can-shaped glass case in an atmosphere of a high-purity nitrogen gas having a purity of 99.999% or more, and an electrode extraction wiring was arranged to fabricate an organic EL element 4-1.

<Fabrication of Organic EL Elements 4-2 to 4-4>

Organic EL elements 4-2 to 4-4 were each fabricated in the same manner as in the fabrication of organic EL element 4-1 except that comparative compound 2 was changed to the compounds shown in Table V.

«Evaluation of Organic EL Elements 4-2 to 4-»

When evaluating the obtained organic EL elements, organic EL elements 4-2 to 4-4 were sealed in the same manner as organic EL elements 1-1 to 1-3 of Example 1, and lighting devices as shown in FIGS. 1 and 2 were each fabricated and evaluated.

Each sample fabricated in this manner was subjected to evaluation, as in Example 2, of the initial film density, the ratio of change in density after storage, the external extraction quantum efficiency, the half-life, the drive voltage and the voltage rise upon drive, the vapor deposition reproducibility, the presence or absence of burning of the boat. The measurement results of the ratio of change in density after storage, the external extraction quantum efficiency, the half-life, the drive voltage, and the voltage rise upon drive in Table V were shown as relative values with respect to the measured values of organic EL element 4-1 as 100.

TABLE 5
Table V

| Organic | | | Ratio of | | | | | | | |
|---|---|---|---|---|---|---|---|---|---|---|
| EL | | Initial film | change in | | | | Change | | Presence or | |
| element | | density | film density | Drive | Luminous | Element | in drive | | absence of | |
| No. | Compound | [g/cm³] | after storage | voltage | efficiency | life | voltage | Reproducibility | burning | Remarks |
| 4-1 | Comparative | 1.1720 | Unacceptable | 100 | 100 | 100 | 100 | Unacceptable | Fair | Comparative |
| | Compound 2 | | | | | | | | | Example |
| 4-2 | Exemplified | 1.0605 | Good | 91 | 111 | 164 | 91 | Good | Good | Present |
| | Compound 9 | | | | | | | | | invention |
| 4-3 | Exemplified | 1.0974 | Good | 79 | 132 | 223 | 81 | Good | Good | Present |
| | Compound 10 | | | | | | | | | invention |
| 4-4 | Exemplified | 1.0793 | Good | 83 | 128 | 259 | 78 | Good | Good | Present |
| | Compound 11 | | | | | | | | | invention |

As is clear from Table V, when using the compound according to the present invention for the element fabrication by vapor deposition, decomposition upon heating did not occur, and the elements could be fabricated with satisfactory reproducibility. Moreover, it was found that the initial film density was small and the ratio of change in film density after storage was remarkably small.

Furthermore, it was clear that the luminous efficiency and light emission life were superior to those of the organic EL element of the Comparative Examples and the drive voltage was low, and the voltage rise upon drive was found to be inhibited.

Similarly, the burning of the resistance heating boat was small.

### Example 6

### <Fabrication of Organic EL Element 5-1>

Patterning was carried out on a substrate (NA45 manufactured by AvanStrate Inc.) in which ITO (indium tin oxide) having a thickness of 100 nm was vapor-deposited on a glass substrate having a size of 100 mm×100 mm×1.1 mm as an anode. Then, the transparent support substrate arranged with the ITO transparent electrode was ultrasonically cleaned with isopropyl alcohol, dried with a dry nitrogen gas, and washed with UV ozone for 5 minutes.

This transparent support substrate was fixed to the substrate holder of a commercially available vacuum vapor deposition apparatus, the vacuum chamber was decompressed to 4×10⁻⁴ Pa, and then the heating boat containing HAT-CN was energized and heated, and the vapor deposition was carried out at the vapor deposition rate of 0.1 nm/s to form a hole injection layer having a layer thickness of 10 nm on a transparent support substrate.

Further, the heating boat containing TBB was energized and heated, and vapor deposition was carried out at the vapor deposition rate of 0.1 nm/sec to form a hole transport layer having a layer thickness of 30 nm on the hole injection layer.

Further, the heating boats containing F-2, Ir-1, Ir-2 and host 2 were energized and heated, and they were vapor co-deposited on the hole transport layer at the vapor deposition rates of 0.08 nm/sec and 0.92 nm/sec, respectively, to form a light emitting layer having a layer thickness of 30 nm.

Furthermore, the heating boat containing comparative compound 4 was energized and heated, but it could not be vapor-deposited.

<Fabrication of Organic EL Element 5-2>

Organic EL element 5-2 was fabricated in the same manner as in the fabrication of organic EL element 5-1 up to the formation of the light emitting layer.

Further, the heating boat containing comparative compound 3 was energized and heated, and vapor deposition was carried out at the vapor deposition rate of 0.1 nm/sec to form an electron transport layer having a layer thickness of 50 nm on the light emitting layer.

Further, after forming lithium fluoride with a film thickness of 1 nm, aluminum having a thickness of 100 nm was vapor-deposited to form a cathode.

The non-light emitting surface side of the above element was covered with a can-shaped glass case in an atmosphere of a high-purity nitrogen gas having a purity of 99.999% or more, and an electrode extraction wiring was arranged to fabricate an organic EL element 5-2.

### <Fabrication of Organic EL Elements 5-3 to 5-6>

Organic EL elements 5-3 to 5-6 were each fabricated in the same manner as in the fabrication of organic EL element 5-2 except that comparative compound 3 was changed to the compound shown in Table VI.

«Evaluation of Organic EL Elements 5-2 to 5-6»

When evaluating the obtained organic EL elements, organic EL elements 5-2 to 5-6 were sealed in the same manner as organic EL elements 1-1 to 1-3 of Example 1, and lighting devices as shown in FIGS. 1 and 2 were each fabricated and evaluated.

Each sample fabricated in this manner was subjected to evaluation, as in Example 2, of the initial film density, the ratio of change in density after storage, the external extraction quantum efficiency, the half-life, the drive voltage and the voltage rise upon drive, and the vapor depositionability (if vapor deposition is possible, it is defined as “Possible”), the vapor deposition reproducibility, and the presence or absence of burning of the boat. The measurement results of the ratio of change in density after storage, the external extraction quantum efficiency, the half-life, the drive voltage, and the voltage rise upon drive in Table VI were shown as relative values with respect to the measured value of organic EL element 5-2 as 100.

TABLE 6
Table VI

| Organic | | | Ratio of | | | | |
|---|---|---|---|---|---|---|---|
| EL | | Initial film | change in | | | | |
| element | | density | film density | Molecular | Drive | Luminous | Element |
| No. | Compound | [g/cm³] | after storage | weight | voltage | efficiency | life |
| 5-1 | Comparative | 1.0943 | Unacceptable | 942.17 | — | — | — |
| | Compound 4 | | | | | | |
| 5-2 | Comparative | 1.0944 | Unacceptable | 1231.44 | 100 | 100 | 100 |
| | Compound 3 | | | | | | |
| 5-3 | Exemplified | 1.0891 | Good | 1165.29 | 95 | 110 | 145 |
| | Compound 12 | | | | | | |
| 5-4 | Exemplified | 1.1078 | Good | 1075.21 | 89 | 119 | 187 |
| | Compound 13 | | | | | | |
| 5-5 | Exemplified | 1.0872 | Good | 1622.87 | 76 | 129 | 245 |
| | Compound 14 | | | | | | |
| 5-6 | Exemplified | 1.0724 | Good | 1010.14 | 73 | 135 | 312 |
| | Compound 15 | | | | | | |

| Organic | | | | | |
|---|---|---|---|---|---|
| EL | Change | | | Presence or | |
| element | in drive | Vapor | | absence of | |
| No. | voltage | depositionability | Reproducibility | burning | Remarks |
| 5-1 | — | Unacceptable | — | — | Comparative |
| | | | | | Example |
| 5-2 | 100 | Good | Fair | Unacceptable | Comparative |
| | | | | | Example |
| 5-3 | 93 | Good | Good | Good | Present |
| | | | | | invention |
| 5-4 | 89 | Good | Good | Good | Present |
| | | | | | invention |
| 5-5 | 81 | Good | Good | Good | Present |
| | | | | | invention |
| 5-6 | 72 | Good | Good | Good | Present |
| | | | | | invention |

As is clear from Table VI, when using the compound according to the present invention for the element fabrication by vapor deposition, decomposition upon heating did not occur, and the elements could be fabricated with good reproducibility. Moreover, it was found that the initial film density was small and the ratio of change in film density alter storage was remarkably small.

Furthermore, it was clear that the luminous efficiency and light emission life were superior to those of the organic EL element of the Comparative Examples and the drive voltage was low, and the voltage rise upon drive was found to be inhibited.

Similarly, the burning of the resistance heating boat was found to be small.

### Example 7

### <Fabrication of Organic EL Element 6-1>

Patterning was carried out on a substrate (NA45 manufactured by AvanStrate Inc.) in which ITO (indium tin oxide) having a thickness of 100 nm was vapor-deposited on a glass substrate having a size of 100 mm×100 mm×1.1 mm as an anode. Then, the transparent support substrate arranged with the ITO transparent electrode was ultrasonically cleaned with isopropyl alcohol, dried with a dry nitrogen gas, and washed with UV ozone for 5 minutes.

This transparent support substrate was fixed to the substrate holder of a commercially available vacuum vapor deposition apparatus, the vacuum chamber was decompressed to 5×10⁻⁴ Pa, and then the heating boat containing HAT-CN was energized and heated, and the vapor deposition was carried out at the vapor deposition rate of 0.1 nm/sec to form a hole injection layer having a layer thickness of 10 nm on a transparent support substrate.

Further, the heating boat containing TAPC was energized and heated, and vapor deposition was carried out at the vapor deposition rate of 0.1 nm/sec to form a hole transport layer having a layer thickness of 30 nm on the hole transport layer.

Further, the heating boat containing mCP was energized and heated, and vapor deposition was carried out at the vapor deposition rate of 0.1 nm/sec to form an electron blocking layer having a layer thickness of 10 nm on the hole transport layer.

Further, the heating boats containing exemplified compound 10 and PPT were energized and heated, and they were vapor co-deposited on the electron blocking layer at the vapor deposition rates of 0.15 nm/sec and 0.75 nm/sec, respectively, to form a light emitting layer having a layer thickness of 30 nm.

Further, the healing boat containing PPT was energized and heated, and vapor deposition was carried out at the vapor deposition rate of 0.1 nm/sec to form an electron transport layer having a layer thickness of 40 nm on the light emitting layer.

Further, after forming lithium fluoride with a film thickness of 0.8 nm, aluminum having a thickness of 100 nm was vapor-deposited to form a cathode.

The non-light emitting surface side of the above element was covered with a can-shaped glass case in an atmosphere of a high-purity nitrogen gas having a purity of 99.999% or more, and an electrode extraction wiring was arranged to fabricate an organic EL element 6-1.

### Fabrication of Organic EL Elements 6-2 to 6-6>

Organic EL element 6-2 was fabricated in tile same manlier as in the fabrication of organic EL element 6-1 except that the heating boat containing exemplified compound 10 and PPT were energized and heated, and they were vapor co-deposited on the light emitting layer at the vapor deposition rates of 0.20 nm/sec and 0.80 nm/sec, respectively, to form an electron transport layer having a layer thickness of 40 nm. In this case, the concentration of the exemplified compound was set to 20% by weight. Organic EL elements 6-3 to 6-6 were each fabricated in the same manner except that tile electron transport layer was formed so as to have the concentration shown in Table VII in the fabrication of organic EL elements 6-3 to 6-6.

«Evaluation of Organic EL Elements 6-1 to 6-6»

When evaluating the obtained organic EL elements, organic EL elements 6-1 to 6-6 were sealed in the same manner as organic EL elements 1-1 to 1-3 of Example 1, and lighting devices as shown in FIGS. 1 and 2 were each fabricated and evaluated.

Each sample fabricated in this manner was subjected to evaluation, as in Example 2, of the external extraction quantum efficiency, the half-life, the drive voltage and the voltage rise upon drive, and the vapor deposition reproducibility. The measurement results of the ratio of change in density after storage, the external extraction quantum efficiency, the half-life, the drive voltage, and the voltage rise upon drive in Table VII were shown as relative values with respect to the measured values of organic EL element 6-1 as 100.

TABLE 7
Table VII

| Organic | | | | | | | | |
|---|---|---|---|---|---|---|---|---|
| EL | | Concentration | | | | Change | | |
| element | | in film | Drive | Luminous | Element | in drive | | |
| No. | Compound | [% by weight] | voltage | efficiency | life | voltage | Reproducibility | Remarks |
| 6-1 | Exemplified | 0 | 100 | 100 | 100 | 100 | Fair | Comparative |
| | Compound 10 | | | | | | | Example |
| 6-2 | Exemplified | 20 | 95 | 111 | 131 | 92 | Fair | Present |
| | Compound 10 | | | | | | | invention |
| 6-3 | Exemplified | 50 | 89 | 132 | 176 | 85 | Good | Present |
| | Compound 10 | | | | | | | invention |
| 6-4 | Exemplified | 75 | 86 | 143 | 187 | 79 | Good | Present |
| | Compound 10 | | | | | | | invention |
| 6-5 | Exemplified | 96 | 81 | 151 | 209 | 71 | Good | Present |
| | Compound 10 | | | | | | | invention |
| 6-6 | Exemplified | 100 | 80 | 159 | 256 | 68 | Good | Present |
| | Compound 10 | | | | | | | invention |

As is clear from Table VII, when using the compound according to the present invention for the element fabrication by vapor deposition, decomposition upon heating did not occur, and the elements could be fabricated with satisfactory reproducibility. Furthermore, it was clear that the luminous efficiency and the light emitting life were excellent and the drive voltage was low by increasing the concentration in the film as compared with comparative organic EL element 6-1 that does not use exemplified compound 10, and the voltage rise upon device was found to be inhibited.

### Example 8

A vapor deposition start temperature of each aromatic compound according to samples a to d shown in the table below was measured by using a saturated vapor pressure measuring apparatus VPE-9000 (manufactured by ADVANCE RIKO, Inc.). About 10 mg of the sample was placed on a pan, the inside of the apparatus was decompressed to a degree of vacuum of 1.5×10⁻² Pa, and then the change in weight by heating was measured at a rate of elevating temperature rate of 20° C./10 minutes while keeping the decompression. The intersection of the tangent of the curve at the temperature at which the slope of the weight reduction curve was maximized, and the initial weight value, was defined as the vapor deposition start temperature.

The vapor deposition start temperature measured in this way and the molecular weight of each compound are shown in Table VIII below.

[Table 8]

TABLE VIII

| | | | Vapor | |
|---|---|---|---|---|
| | | | deposition | |
| | | | start | |
| Sample | | Molecular | temperature | |
| No. | Compound | weight | [° C.] | Remarks |
| a | Comparative | 1058 | 463 | Comparative |
| | Compound 5 | | | Example |
| b | Exemplified | 1233 | 392 | Present |
| | Compound 7 | | | invention |
| c | Exemplified | 929 | 336 | Present |
| | Compound 9 | | | invention |
| d | Exemplified | 934 | 342 | Present |
| | Compound 10 | | | invention |

As is clear from Table VIII, the aromatic compound according to the present invention can inhibit the increase in the vapor deposition temperature even if the molecular weight is 900 or more. in particular even if the molecular weight exceeds 1,000, the element can be fabricated by the vapor deposition, so that the element having a high Tg and high stability can be fabricated.

### INDUSTRIAL APPLICABILITY

The functional film of the present invention that is excellent in low voltage driveability, high luminous efficiency, long service life, resistance to drive voltage fluctuation and vapor deposition reproducibility, is suitably used for a light emitting device for displays or a lighting device, for example, as an organic EL light emitting device.

### REFERENCE SIGNS LIST

101 Organic EL element

102 Glass cover

105 Cathode

106 Organic EL layer

107 Glass substrate with transparent electrode

108 Nitrogen gas

109 Water-collecting agent

1. A functional film comprising an aromatic compound, wherein
the aromatic compound has a condensed or noncondensed 6-membered aromatic hydrocarbon ring or aromatic heterocyclic ring having four or more condensed aromatic ring groups containing not less than 14Π electrons, wherein three or more of the condensed aromatic ring groups containing not less than 14Π electrons are adjacent to one another as substituents, and wherein
for the aromatic compound, when a film density value calculated by molecular dynamics calculation of NPT ensemble at 300 K is defined as an initial film density of the functional film comprising only the aromatic compound, and
for the aromatic compound, when a film density value calculated by molecular dynamics calculation at 370 K is defined as a film density value after storage of the functional film at the temperature, the difference between the initial film density and the film density value after storage is 1% or less with respect to the initial film density.

2. The functional film according to claim 1, wherein the initial film density value is in the range of 1.00 to 1.20 g/cm³.

3. The functional film according to claim 1, wherein the aromatic compound has a chirality generation site.

4. The functional film according to claim 1, wherein the aromatic compound has a structure represented by the following general formula (1):
Ar [symbol] HetAr)ₙ General formula (1)
wherein Ar represents substituted or unsubstituted heteroaryl, or substituted or unsubstituted aryl; HetAr represents a substituted or unsubstituted condensed aromatic heterocyclic group containing not less than 14Π electrons; and n represents an integer of 4 or more.

5. The functional film according to claim 1, wherein the aromatic compound has a structure represented by the general formula (2): 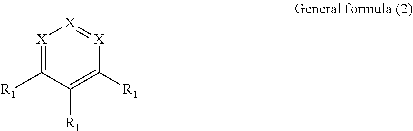
wherein X represents N or CR₂ and at least one X represents CR₂; R₁ represents a condensed aromatic heterocyclic group containing not less than 14Π electrons; and R₂ represents a hydrogen atom or R₁, or represents any group selected from the group consisting of an alkyl group, a cycloalkyl group, an alkenyl group, an aromatic hydrocarbon group, an aromatic heterocyclic group, a heterocyclic group, an alkoxy group, a cycloalkoxy group, an aryloxy group, an alkylthio group, a cycloalkylthio group, an arylthio group, an aryloxycarbonyl group, a sulfamoyl group, an alkoxycarbonyl group, an acyl group, an acyloxy group, an amide group, a carbamoyl group, a ureido group, a sulfinyl group, an alkylsulfonyl group, an arylsulfonyl group or a heteroarylsulfonyl group, an amino group, a halogen atom, a fluorinated hydrocarbon group, a cyano group, a nitro group, a hydroxy group, a mercapto group, a silyl group, and a phosphono group, provided that at least one R₂ represents R₁.

6. The functional film according to claim 1, wherein the condensed aromatic heterocyclic group containing not less than 14Π electrons has a nitrogen (N) atom.

7. The functional film according to claim 1, wherein the condensed aromatic heterocyclic group containing not less than 14Π electrons has at least two nitrogen (N) atoms.

8. The functional film according to claim 1, wherein the aromatic compound has five or less condensed aromatic ring groups containing not less than 14Π electrons.

9. The functional film according to claim 5, wherein in the compound having the structure represented by the general formula (2), R₂ represents R₁, or represents any group selected from the group consisting of a cycloalkyl group, an aromatic hydrocarbon group, an aromatic heterocyclic group, an amino group, a fluorinated hydrocarbon group, a nitro group, a silyl group and a phosphono group.

10. The functional film according to claim 1, wherein the aromatic compound has a molecular weight in the range of 1,000 to 2,000.

11. The functional film according to claim 1, comprising the aromatic compound in an amount of 50% by weight or more.

12. The functional film according to claim 1, being a charge transport film.

13. A method for forming the functional film according to claim 1 by a vacuum vapor deposition method.

14. An organic electroluminescence element comprising at least a pair of electrodes and one or a plurality of layers, wherein at least one layer of the plurality of layers has the functional film according to claim 1.

15. The organic electroluminescence element according to claim 14, wherein at least two adjacent layers among the plurality of layers are each the functional film.
